(19) United States
(12) Patent Application Publication
(10) Pub. No.: US 2003/0229394 A1
(43) Pub. Date: Dec. 11, 2003

(54) Processed tissue for medical device formation

(76) Inventors: Matthew F. Ogle, Oronoco, MN (US); Steven D. Kruse, Bloomington, MN (US)

Correspondence Address: ALTERA LAW GROUP, LLC, 6500 CITY WEST PARKWAY, SUITE 100, MINNEAPOLIS, MN, 55344-7704, US

(21) Appl. No.: 10/164,725

(22) Filed: Jun. 6, 2002

(51) Int. Cl. A61F 2/24

(52) U.S. Cl. 623/2.14; 623/918; 8/94.11

(57) ABSTRACT

In some embodiments, a method for processing tissue comprises the application of a directional load to modify the properties of the tissue. In particular, the directional force is sufficient to increase the rigidity of the tissue asymmetrically relative to an unaligned tissue equivalently processed without being subjected to a load. In some embodiments, a sufficient directional load is applied to increase the rigidity of the tissue relative to an unaligned tissue equivalently processed that is not subjected to the load, in which the load is applied with a load applicator. A connector transfers the load from the load applicator to the tissue. Selectively aligned tissue having asymmetric mechanical properties can be used to form a prosthetic valve. The leaflets are matched with respect to each of their properties to have improved coaptation relative to corresponding tissue leaflets with symmetrical mechanical properties.

### FIELD OF THE INVENTION

The invention relates a process to modify tissue relative to bending and in plane extensibility about axes perpendicular to the aligned direction. The invention further relates to medical devices, especially valved prostheses, formed from tissue that is artificially aligned and corresponding methods for forming valved prostheses. In addition, the invention relates to apparatuses for aligning tissue and corresponding methods for aligning tissue.

### BACKGROUND OF THE INVENTION

Various medical articles have been designed particularly for contact with a patient's body fluids. This contact can be sufficiently long such that surface interactions between the medical article and the patient's blood and/or tissue become significant. For example, the interaction of blood with the surface of the medical article can lead to degradation, such as calcification of the medical article. Relevant medical articles include, for example, catheters and prostheses.

Prostheses, i.e., prosthetic devices, are used to repair or replace damaged or diseased organs, tissues and other structures in humans and animals. Prostheses generally are biocompatible since they are typically implanted for extended periods of time. Prostheses can be constructed from natural materials, synthetic materials or a combination thereof.

Bioprosthetic heart valves from natural materials were introduced in the early 1960's. Bioprosthetic heart valves typically are derived from pig aortic valves or are manufactured from other biological materials, such as bovine pericardium. Xenograft heart valves are typically fixed with glutaraldehyde or other crosslinking agents prior to implantation to reduce the possibility of immunological rejection. Glutaraldehyde reacts to form covalent bonds with free functional groups in proteins, thereby chemically crosslinking nearby proteins.

The importance of bioprosthetic heart valves as replacements for damaged human heart valves has resulted in a considerable amount of interest in the design, formation and long term performance of these valves. In particular, the character of natural tissues poses issues that are not faced with respect to most synthetic materials. For example, quality control and uniformity of the raw materials are not as easy to control for natural materials. When assembling bioprosthetic heart valves from segments of tissue, structural irregularities in the tissue can complicate the process and make the process less reproducible. A low level of reproducibility can result in waste of tissue and added expense.

### SUMMARY OF THE INVENTION

In a first aspect, the invention pertains to a method for processing tissue. The method comprises applying a sufficient directional load to the tissue to increase the rigidity of the tissue asymmetrically relative to tissue equivalently processed that is not subjected to the load.

In a further aspect, the invention pertains to a method for processing a tissue. The method comprises applying a sufficient load to the tissue to increase the rigidity of the tissue relative to tissue equivalently processed that is not subjected to the load. A load applicator applies the load to the tissue. Also, a connector transfers load from the load applicator to the tissue.

In another aspect, the invention pertains to a method for forming a prosthetic valve. The method comprises assembling a plurality of tissue leaflets to form the valve. The tissue leaflet comprises selectively aligned tissue having asymmetric mechanical properties.

In addition, the invention pertains to biocompatible tissue comprising selectively aligned tissue having an asymmetric flexibility. The tissue comprises pericardial tissue, amniotic sac tissue, blood vessel tissue, cartilage, dura mater tissue, skin tissue, fascia tissue, submucosa tissue, or umbilical tissue.

Furthermore, the invention pertains to a prosthetic valve comprising a plurality of tissue leaflets. The tissue leaflets comprise selectively aligned tissue.

Also, the invention pertains to an apparatus comprising tissue and a load applicator that applies a selected mechanical load to the tissue. The load applicator comprises a gripper that grips adjacent a selected subsection of the edge of the tissue that does not approximate gripping the edge equally around the tissue.

### BRIEF DESCRIPTION OF THE DRAWINGS

FIG. 1 is a side perspective view of a three leaflet, stentless heart valve prosthesis.

FIG. 2 is a side perspective view of a four leaflet, stentless mitral heart valve prosthesis.

FIG. 3 is a side perspective view of a three leaflet stented heart valve prosthesis.

FIG. 4A is a perspective view of a vascular prosthesis.

FIG. 4B is a side view of the vascular prosthesis of FIG. 4A attached to blood vessels.

FIG. 5 is a schematic perspective view of a tensioning apparatus for aligning tissue.

FIG. 6 is a schematic perspective view of an embodiment of a tensioning device comprising a spiked frame for holding tissue under tension.

FIG. 7 is a perspective view of a gripper connected to an anchor.

FIG. 8 is a top view of four connectors that join to a single connector leading to a load applicators.

FIG. 9 is a top view of three connectors leading to three separate load applicators for attachment to a side of a tissue element.

FIG. 10 is a side view of a connector attached to a weight.

FIG. 11 is a side view of an embodiment supporting tissue in a vertical configuration with a weight functioning as a load applicator.

FIG. 12 is a side view of a motor attached to a connector, in which the motor functions as a load applicator.

FIG. 13 perspective view of a tissue element attached to the top of a hollow cylinder in which tension is applied with a contact probe.

FIG. 14 is side view of an adjustable load applicator based on a spring.

FIG. 15 is a perspective view of a tray with fluid for immersing tissue during alignment of the tissue.

FIG. 16 is a schematic perspective view of a moisture source including two spray nozzles.

FIG. 17 is a schematic perspective view of a tissue element within an enclosure with a reservoir of liquid serving as the moisture source.

FIG. 18 is a side view of a tissue element on a curved tissue support with load applied from above.

FIG. 19 is a fragmentary top perspective view of the tissue support of FIG. 18.

FIG. 20 is side view of a leaflet section for introduction into the prosthesis of FIG. 1.

FIG. 21 is a side view of a post segment for incorporation into the prosthesis of FIG. 1.

FIG. 22 is a side view of a bias strip for incorporation into the prosthesis of FIG. 1.

FIG. 23 is a side view of a first leaflet section for the four leaflet heart valve prosthesis of FIG. 2.

FIG. 24 is a side view of a second leaflet section for the four leaflet heart valve prosthesis of FIG. 2.

FIG. 25 is a side view of a third leaflet section for the four leaflet heart valve prosthesis of FIG. 2.

FIG. 26 is a top view of a fourth leaflet section for the four leaflet heart valve prosthesis of FIG. 2.

FIG. 27 is a side perspective view of a stent and a leaflet from the prosthesis of FIG. 3.

FIG. 28 is a side perspective view of the leaflet of FIG. 27 partially attached to the stent.

FIG. 29 is a side perspective view of the stent of FIG. 27 with two leaflets partially attached to the stent.

FIG. 30 is a schematic representation of a flexibility grading scale based on the placement of a tissue disk onto a rod.

FIG. 31 is a side perspective view of an apparatus for aligning tissue in a vertical orientation using a weight.

### DETAILED DESCRIPTION OF THE INVENTION

The selective alignment of properties of tissue, such as stiffness, other mechanical properties and/or morphology, can lead to advantageous and more reproducible performance of the tissue. The selective alignment can result in tissue with mechanical properties corresponding more closely to properties in the corresponding native tissue structure to be replaced with the tissue and/or with more consistent properties appropriate for the particular use. Without being bound by theory, the alignment of the tissue properties may result in the alignment of collagen fibrils and possibly other rigid structural proteins, such as elastin. In some embodiments, the properties of an element of tissue can be artificially aligned by applying a load or force to induce stress, for example, by pulling at opposite edges of a sheet of tissue. In some embodiments, the tissue has contours forming a non-planar structure. For convenience, tissue with aligned properties may be referred to herein as aligned tissue, and the process of aligning tissue properties may be referred herein to as alignment of tissue.

Appropriate apparatuses for selectively aligning tissue can grip the tissue or tissue element and apply a suitable amount of force to perform the desired degree of alignment. The selective alignment can be performed prior to or during any crosslinking of the tissue. The selectively aligned tissue can be assembled into medical devices, especially implantable medical devices. In particular, tissue used in prostheses, such as pericardium, can be selectively aligned to more closely resemble heart valve leaflet tissue of a native heart valve such that the pericardial tissue performs more similarly to a native heart valve leaflet and has properties that are more consistent between different samples. In addition, the tissue can be made more uniform since the native tissue may have uneven alignment prior to processing. For example, native pericardium may be mostly unaligned with small areas of alignment. Thus, a random sampling of pericardium can have relatively unpredictable properties.

In general, relevant medical devices are bioprostheses that are formed to mimic a corresponding structure within the body, although suitable medical devices can be percutaneous devices with long-term contact with body fluids. Bioprostheses can be used to replace or repair the corresponding native structure. The prosthetic devices generally are suitable for long-term implantation within a recipient patient. In embodiments of particular interest, the patient is an animal, preferably a mammal, such as a human. The medical devices generally include at least a component that is formed from a tissue. A component of the medical device with a tissue can have specific mechanical requirements for a desired function within the medical device. The properties of the tissue may have to be consistent with the particular use of the tissue.

The selectively aligned bioprosthetic tissue can be used in valved prostheses, especially heart valve prostheses. Damaged or diseased native heart valves can be replaced with valved prostheses to restore valve function. Heart valve prostheses of interest have leaflets formed from tissue. Heart valve prostheses with tissue leaflets can be designed as a replacement for any heart valve, i.e., an aortic valve, a mitral valve, a tricuspid valve, or a pulmonary valve. In addition, valved prostheses with leaflets formed from selectively aligned tissue can be used for the replacement of vascular valves.

In a valve with tissue leaflets, the leaflets flex to open and close the valve. The leaflets are supported by a support structure that includes commissure supports and scallops extending between the commissure supports. The commissure supports hold the ends of the free edge of the leaflets. Commissure supports may or may not extend beyond the attachment points of the leaflet in the flow direction. The attached edge of the leaflet is located along scallops and ends at the commissure support. The attached edges of adjacent leaflets approach each other at the commissure support. The support structure of the valve may comprise a sewing cuff or the like for attachment of the valve to the patient's annulus, to other components of a medical device, or anatomical structure.

In some embodiments, the support structure comprises a rigid component that maintains the leaflet function of the valve against the forces opening and closing the valve. Valves with a rigid support structure are termed stented valves, and the rigid support is called a stent. The stent provides a scaffolding for the leaflets. The stent generally is sufficiently rigid such that only the base of the stent is attached to the patient or other device. As a particular example, heart valve stents are used to support leaflet components within a prosthetic heart valve.

In alternative embodiments, the support structure is not sufficiently rigid to maintain the leaflet function of the valve against the forces opening and closing the valve. In these embodiments, the valve is termed stentless. In a stentless valve, the support structure also has commissure supports at which the free edge of the leaflet connects with the support structure, and scallops which support the attached edge of the leaflets. However, in the stentless valve, the support structure is less rigid such that both edges of the support structure, i.e., the inflow edge and the outflow edge, must be secured such as by suturing or other fastening approach to other anatomical structures, such as the wall of a blood vessel, or to other device structures to prevent the valve from collapsing against the fluid pressure. The support structure can be formed from tissue or from other flexible material or materials in a configuration that defines the commissure supports and the scallops or other suitable interface that hold the attached edges of the leaflet.

The valve generally includes a plurality of leaflets. In particular, generally the valves function as one way check valves that open to allow flow in a desired direction and close in response to pressure differentials to limit reverse flow. Thus, during forward blood flow, the leaflets fully open to allow for flow through the valve. In the open position, the free edges of the tissue leaflets form the downstream opening of the valve and generally do not significantly resist forward blood flow.

When the valve closes in response to pressure differentials, the free edges of adjacent leaflets contact in a closed position with the leaflets extending across the lumen of the valve. The contact of adjacent leaflet free edges across the lumen of the valve eliminates or greatly reduces back flow through the valve. The contacting portion of the leaflets is referred to as the coaptation region.

In general, bioprosthetic valved prostheses with tissue leaflets can be formed from a natural valve or from a tissue assembled into a valve. For example, fixed porcine heart valves can be used to replace damaged or diseased human heart valves. Alternatively, porcine heart valve leaflets can be removed from the native valve and assembled into a valved prosthesis. Native leaflets inherently have appropriate mechanical properties for functioning in a valve unless they have been damaged, although conditions used during crosslinking can affect the mechanical properties. However, the use of other types of bioprosthetic tissue assembled into a bioprosthetic valve provides greater versatility in valve design and availability of materials than with the use of native leaflets. By processing the tissue as described herein, non-cuspal, i.e., non-leaflet, tissue can be made to have properties and corresponding performance more similar to a native leaflet. For example, some valve designs, such as some stented valves that are not designed for formation with native leaflets, can have advantages for implantation over other valve designs.

In native tissue, the degree of alignment of the tissue is correlated with the forces to which the tissue is subjected in the physiological environment and possibly during fixation of a harvested native valve. As described herein, the alignment of the tissue helps the native tissue to perform suitably for the environment, and the physiologic forces themselves experienced by the functioning valve help maintain the alignment of the tissue. Thus, the structure and function of a native tissue tend to mutually reinforce each other. In native tissue, the alignment of the tissue properties appears to be related to the corresponding alignment of fibrous structural proteins, such as collagen. Partial alignment of collagen generally stiffens the tissue with respect to axes that cross more collagen fibrils at angles closer to perpendicular. Alignment of the collagen fibrils along a particular direction can make the mechanical properties of the tissue asymmetric with respect to orientation of the tissue. The alignment can be selected to make the prosthetic tissue more appropriate for a particular use, such as for a valve. Similarly, tissue can be aligned in any direction or over only a portion of the tissue.

In particular, in a leaflet of a native biological valve, such as a heart valve, the tissue, along with the corresponding collagen fibrils, may be aligned. Native leaflets generally can be described as having a three-layered structure, with reach layer having different compositional and mechanical properties. The respective outer layers, the fibrosa layer and the ventricularis layer, have significant amounts of collagen fibrils. The degree of orientation of the collagen fibrils can be affected by the physiological flow conditions experienced by the leaflets. These changes in collagen orientation are described further in Sacks et al., “The aortic valve microstructure: Effects of transvalvular pressure,” J. Biomed. Mater. Res. 41: 131-141 (1998), incorporated herein by reference.

The properties of bioprosthetic tissue can be aligned to mimic performance of native tissue in a particular native structure. For example, for a stented valve, the leaflets can be stiffened such that coaptation of the leaflets is improved. Approximately matching the stiffness of the leaflets, with respect to magnitude of the stiffness, as well as positioning of asymmetric tissue properties, within a single valve leads to better coaptation of the valve. The flow subjects the leaflets to directional stress due to force applied by the fluid when the valve is open. The alignment of the tissue contributes to the flexibility of the leaflets parallel to the flow direction such that the leaflets open properly in response to pressure differentials. Furthermore, this alignment of tissue properties can lead to a decrease in extensibility of the free edge of the leaflet. Less extensible free edges of the leaflets can provide improved leaflet coaptation. While the leaflets can have an appropriate thickness to provide desired levels of mechanical strength, the leaflets can have a higher relative flexibility with respect to opening and closing due to the alignment of the tissue and appropriately orienting the tissue when forming a leaflet. Tissue that lacks alignment can have variation in mechanical properties since alignment can lead to greater structural uniformity. Thus, selected alignment and appropriate orientation of the tissue can lead to more predictable mechanical performance of the tissue by reducing random variations in native unaligned tissue.

Suitable materials for incorporation into prostheses for blood contact are biocompatible, in that they are non-toxic, non-carcinogenic and do not induce hemolysis or a significant immunological response. Heart valve prostheses formed from tissue generally are non-thrombogenic. Relevant mechanical properties of flexible leaflets include, for example, stiffness, strength, creep, hardness, fatigue resistance and tear resistance.

While entire valves can be used to form prosthetic valves and native valve leaflets can be extracted to assemble into prosthetic valves, processing of these structures into prosthetic valves can be difficult to perform without damaging the leaflets and production yields can be low since flawed leaflets cannot be used. In other embodiments, other tissue types, for example, bovine pericardium and fascia can be used to form heart valve leaflets. While native leaflets comprise naturally aligned tissue, other tissues may have less alignment or no alignment. For example, pericardium has randomly oriented collagen fibrils and, thus, generally little alignment since in its function as a protective sac around the heart, the pericardium is not under significant load. Also, pericardium is a multilaminate material that can have varying alignment. The absence of load during pericardial tissue function correlates with the random orientation of the collagen fibrils. The alignment of a tissue can be altered using the approaches described herein to more closely approximate the alignment of the tissue in native heart valve leaflets.

The process of aligning the tissue can be conveniently performed by applying a load to the tissue with sufficient force and an appropriate duration to provide the desired level of alignment, possibly due to partial collagen fibril reorientation. In general, appropriate load can be applied by pulling on opposite sides of a section of tissue or by applying a load in some other configuration. The pulling process also tends to make the section of tissue more planar unless a curved tissue support is used. The stress resulting from the pulling process can result in irreversible changes in the tissue alignment, especially when coupled with crosslinking either during or following the load application. The tissue can be pulled in multiple directions, either sequentially or simultaneously, to align the fibrils in multiple directions, such as two orthogonal directions. In general, for leaflet formation, it is desirable to align the tissue in a single direction, the orientation of which may depend on the overall valve structure, as described further below for two specific embodiments. In some embodiments, it is desirable to use tissue that is aligned in two or more directions that is generally stiffer and has higher tensile strength. For example, such tissue with higher tensile strength is suitable for use as a tendon prosthesis, or a pledget.

The degree of alignment of tissue can be evaluated by measuring the bending of the tissue about a rod or the like. This measurement of flexibility can be used to evaluate alignment of tissue, especially when comparing tissues with similar thicknesses and otherwise similar compositions. In general, tissue having predominantly randomly oriented collagen fibrils, i.e., relatively un-aligned tissue, can be selectively aligned to obtain a desired level of rigidity along a particular direction generally up to some limiting value. Also, tissue with some degree of natural alignment can also be modified using the techniques described herein, but the magnitude of the effect and the limiting value may be different from the corresponding values in un-aligned tissue.

A suitable apparatus for orienting a tissue can comprise a container, an enclosure or the like and a tensioning device. The container/enclosure can support the tissue and can keep the tissue moist during the tissue processing. For example, a container can maintain a sterile solution in which the tissue is immersed during processing. The tensioning device can comprise grippers and a load applicator operably connected to the grippers through connectors. One or more grippers or the like can be used to grip two or more portions of a tissue section. Generally, at least two portions of a tissue section, e.g., two generally opposite sides of a tissue section, such as near an edge, is gripped during the tissue aligning process, although more than two sides can be gripped and stressed by the apparatus. The grippers can be attached to an appropriate load applicator that applies tension by pulling on a gripper. Alternatively, one or more of the grippers can be held in place such that the tissue is maintained in a stressed or tensioned condition. In some embodiments, the tissue can be stretched between a plurality of anchors to block the tissue under tension. In some embodiments, the load applicator can comprise an appropriate mechanical device that may or may not be motorized. For example, the load applicator can comprise an adjustable spring with a selectable tension or a weight that is pulled by gravity. In another example, the load applicator can include a motor with a clutch such that the motor can apply a selected tension.

In general, the tissue can be xenograft, allograft or autograft tissue. The tissue is harvested for processing and may or may not be stored prior to performing the processing. Some preliminary processing can be performed prior to aligning the tissue, such as cutting and trimming of the tissue, sterilizing the tissue, associating the tissue with one or more desirable compositions, such as anticalcification agents and growth factors, and the like. After any preliminary processing and/or storage are completed, the tissue is processed to align the tissue. Following alignment of the tissue, the aligned tissue is further processed, which can involve further chemical and/or mechanical manipulation of the tissue as well as processing the tissue into the desired medical device.

In many embodiments, the tissue is crosslinked prior to forming the medical device. Crosslinking or fixing tissue can be performed, for example, to mechanically stabilize the tissue, decrease or eliminate antigens and/or terminate enzymatic activity. Xenograft tissue, i.e., tissue transplanted between species, generally is crosslinked to eliminate hyper-acute immune response. Crosslinked tissue generally refers to tissue that is fully crosslinked in the sense that further contact with a crosslinking agent does not further change measurable attributes of the tissue. However, the mechanical stabilization of crosslinked tissue generally prevents or at least inhibits alignment of the tissue, possibly due to fixing of the orientation of the collagen fibrils. Therefore, it is desirable to align the tissue prior to forming crosslinked tissue. Forming crosslinked tissue, though, generally requires a significant period of time. Thus, the crosslinking can be performed during the orientation of the tissue as well as after the orientation of the tissue. Partial crosslinking of tissue may not destroy the ability to align the tissue. Therefore, some partial crosslinking of the tissue can be performed prior to the aligning of the tissue.

Following aligning the tissue, processing the tissue into the desired medical device can include, for example, cutting the tissue section to an appropriate size and shape and fastening the cut tissue portions together and/or to other appropriate tissue or non-tissue components. In particular, the aligned tissue can be formed into leaflets for a bioprosthetic valve. The tissue portions are particularly suitable for forming stented valves, although unstented valves can also be formed. Furthermore, the aligned tissue can be advantageously incorporated into biological conduits, as well as other medical devices.

The alignment of a tissue section provides an approach to transform the tissue section to have a structure more similar to an aligned structure of certain native tissue, such as heart valve leaflets. Since the alignment of a tissue can be correlated with functional features of tissue, the aligned tissue can be more suitable for certain applications. Specifically, for tissue that is subjected in the physiological environments to certain loads, the tissue generally is aligned in a corresponding way such that the tissue responds in a desirable way to the loads. Therefore, tissue can be made to respond more like a native tissue even if the tissue is not derived from a similar tissue to the native tissue. The mechanical properties of the tissue therefore can be improved for particular applications. This performance improvement can result in more reproducible tissue characteristics such that waste of tissue can be reduced and a more consistently performing valve is produced. In addition, more predictable and desirable performance can be achieved that is more similar to the performance of native tissue.

Medical Devices

Relevant medical devices generally comprise tissue, at least as a component. In embodiments of particular interest, at least a portion of the tissue included in the medical device is selectively aligned tissue. Generally, these medical devices are prostheses or have components designed for implantation or insertion into or placement onto a patient for extended periods of time. Prostheses include, for example, artificial hearts, artificial heart valves, annuloplasty rings, pericardial patches, vascular and structural stents, vascular grafts or conduits, tendons, pledgets, suture, permanently in-dwelling percutaneous devices, vascular shunts, dermal grafts for wound healing, and surgical patches. Vascular structures include cardiovascular sites and other blood contacting structures. Biomedical devices that are designed to dwell for extended periods of time within a patient are also relevant for modification as described herein.

Medical devices of particular interest include heart valve prostheses, vascular grafts, tendons, annuloplasty rings and patches. The aligned tissue can be incorporated into existing designs or new designs for medical devices assembled from tissue materials. Generally, the selectively aligned tissue is used for prosthetic components that are under a load following implantation. The selectively aligned tissue can be oriented when assembled into the prosthetic device, as described further below, to take advantage of the aligned structure of the tissue.

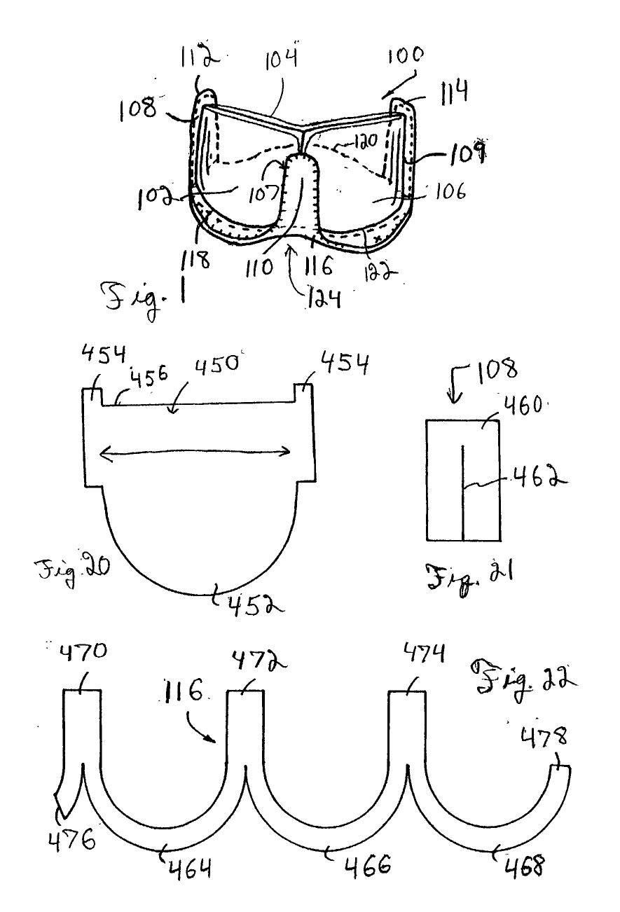

Heart valve prostheses with tissue leaflets can include a stent that serves as a frame for flexible leaflets, or the valve can be stentless, in which a heart valve is implanted utilizing the recipient's native support structure, e.g., the aorta or mitral annulus. As a particular example of a stentless aortic heart valve prosthesis assembled from oriented tissue elements, heart valve prosthesis 100 has three leaflets 102, 104, 106, as shown in FIG. 1. Leaflets 102, 104, 106 are attached to post segments 107, 108, 109 at commissure posts 110, 112, 114. A bias strip 116 forms a wall joining post segments 108 and leaflets 102, 104, 106 along scallops 118, 120, 122 to form a valve structure with an inflow edge 124 at scallops 118, 120, 122.

Another example of a heart valve prosthesis assembled from oriented tissue elements is shown in FIG. 2. A stentless mitral heart valve prosthesis 130 with four leaflets includes a sewing ring 132, and four leaflets 134, 136, 138, 140. Chordae 142 extend from leaflets 134, 136, 138, 140. Chordae 142 and/or associated leaflets can be formed from a single sheet of biocompatible material, such as oriented tissue. Chordae 142 connect with attachment sections 144 for attachment to the patient's papillary muscles. An edge 146 of the tissue forming leaflets 134, 136, 138, 140 is stitched between two portions 148, 150 of sewing ring 132 to secure the leaflets to the sewing ring.

A stented heart valve prosthesis with tissue leaflets is shown in FIG. 3. Stented valve 160 comprises a stent 162, a sewing cuff 164 and three tissue leaflets 166, 168, 170. Stent 162 is made from an appropriate material to prevent the leaflets from collapsing onto themselves when the valve is closed. The tissue is fastened to the stent, for example, as described further below, to secure the tissue in the valve structure. Sewing cuff 164 facilitates implantation by providing a structure for fastening, such as suturing, the valve to native support structure.

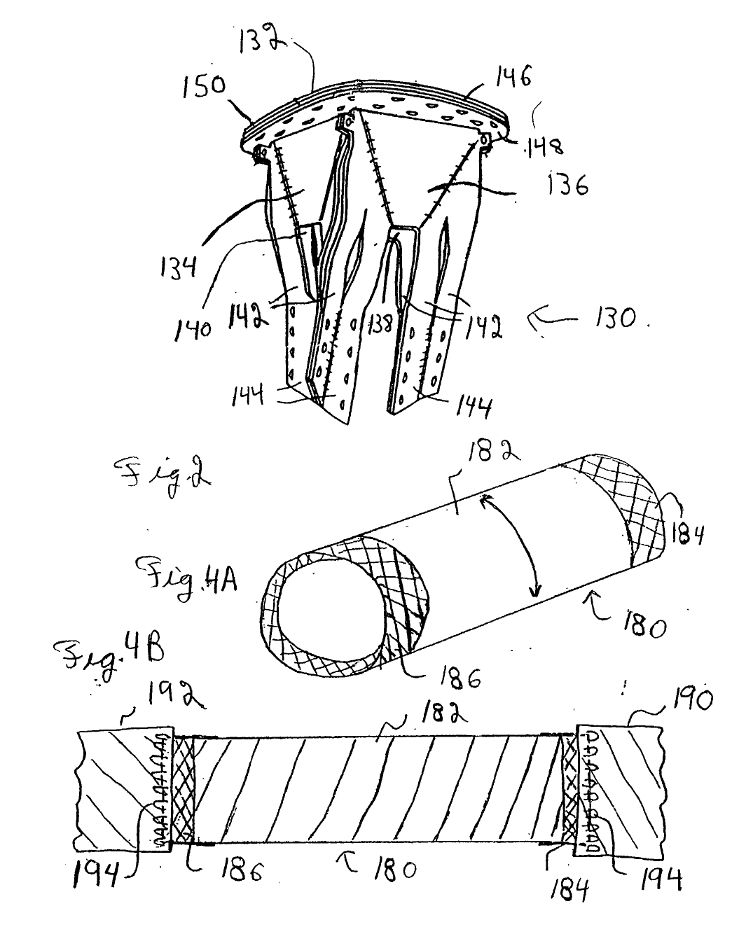

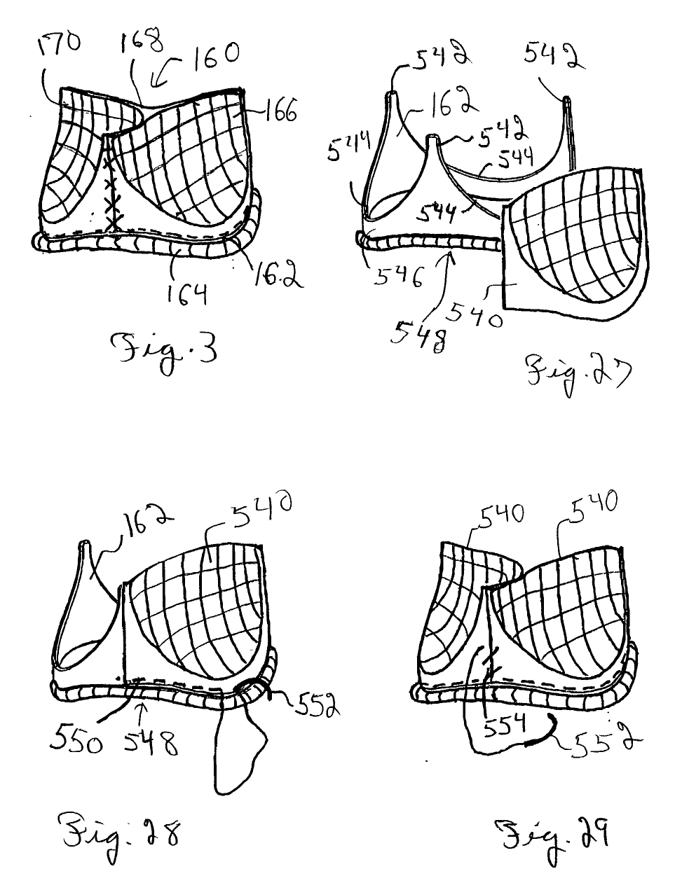

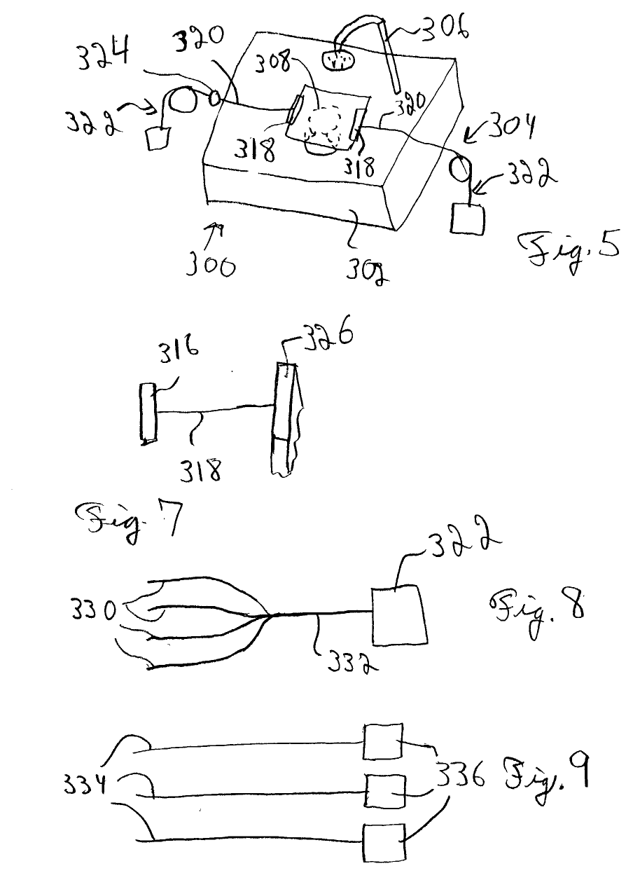

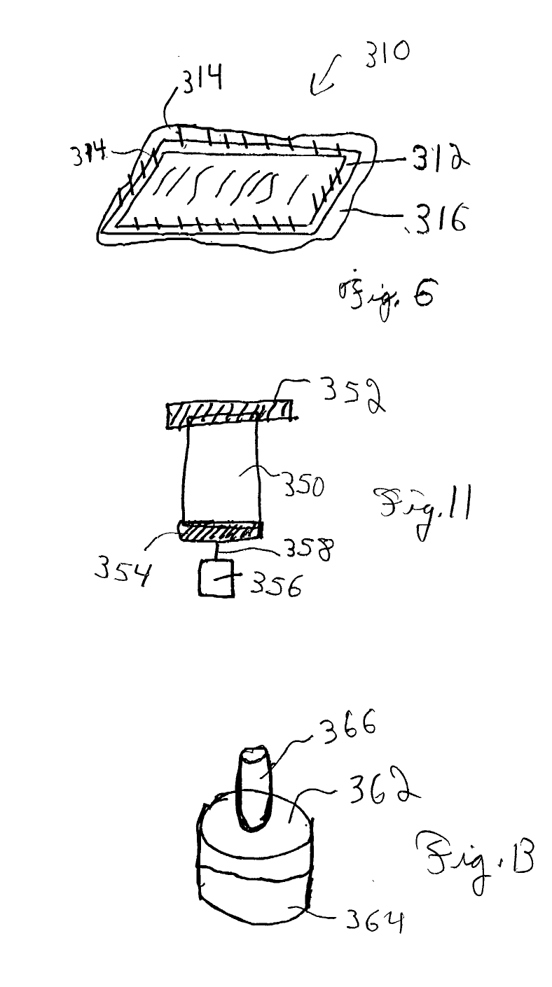

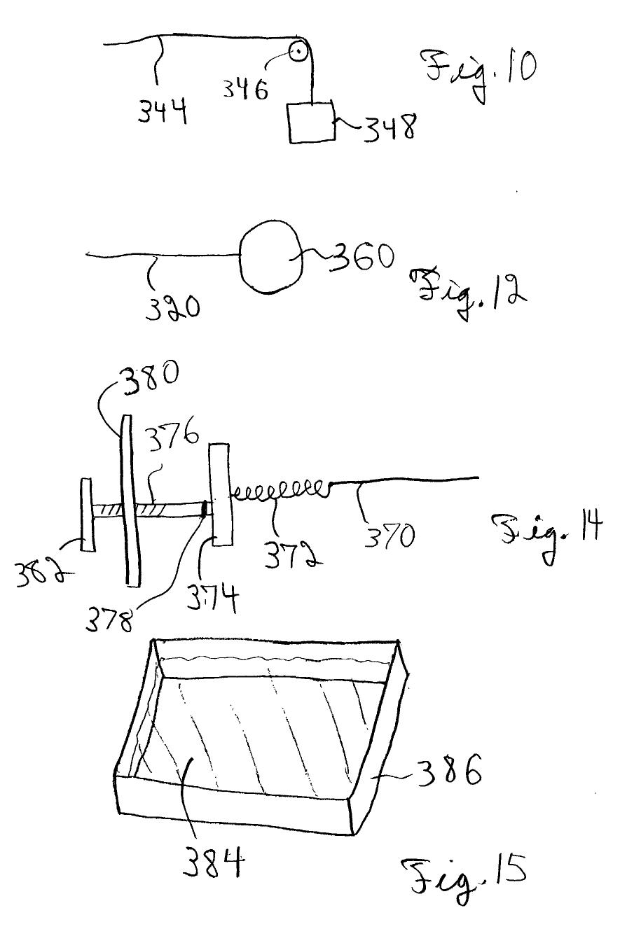

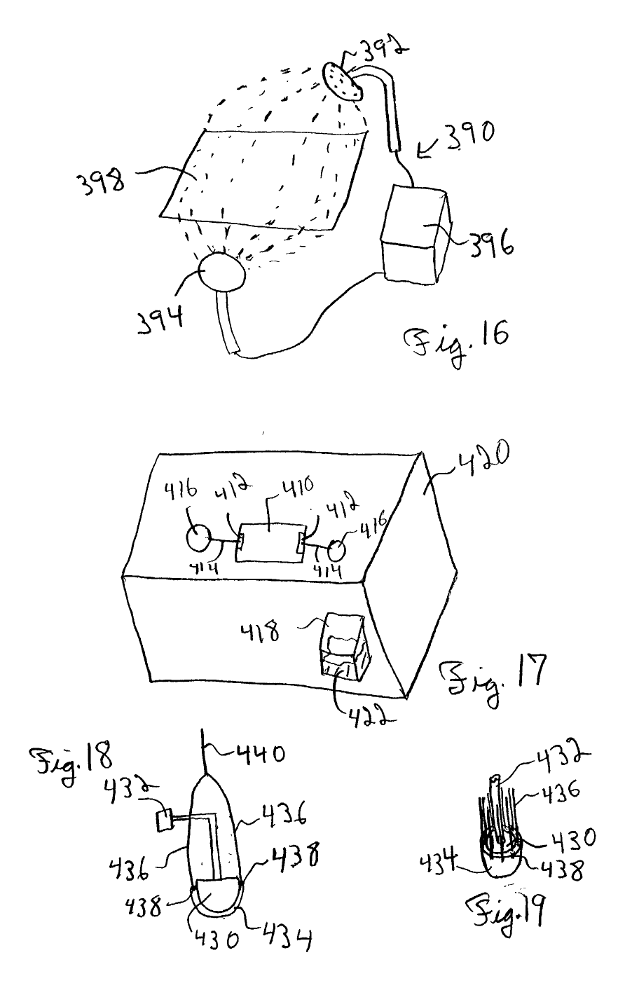

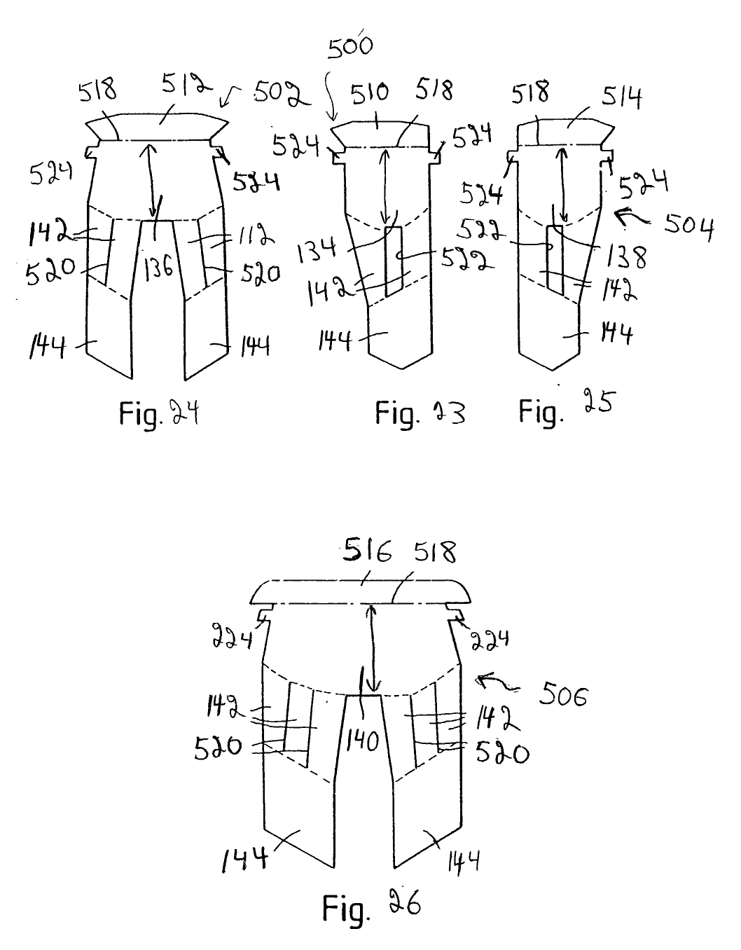

A representative vascular graft 180 is depicted in FIG. 4A. Vascular graft 180 includes a flexible tubular structure 182 and optional sewing cuffs 184, 186. In these embodiments, flexible tubular structure 182 generally comprises tissue, such as selectively aligned tissue. Sewing cuffs 184, 186 can be formed from fabric, tissue or the like. Sewing cuffs 184, 186 assist with the implantation of the prosthesis and may provide reinforcement of the prosthesis at the site of anastomoses, i.e., attachment of the vessel to the graft. A side view of vascular graft 180 attached to natural vessel sections 190, 192 is depicted in FIG. 4B. As shown in FIG. 4B, suture 194 is used to secure vascular graft 180 to vessel sections 190, 192, although other fastening approaches can be used.

Selectively Aligned Tissue

Tissue comprises a protein-based extracellular matrix that generally comprises collagen fibrils usually with other protein and non-protein components. The tissue can be a natural tissue or a synthetic collagen-based matrix. Upon application of an appropriate load, the tissue matrix can align to a selected degree, possibly due to reorientation of collagen fibrils within the tissue matrix. The effectiveness of the load in selectively aligning the tissue (properties) generally depends on the magnitude of the load, the morphology of the tissue, and the length of time that the load is applied. In addition, the effectiveness of alignment of the tissue depends on the initial degree of orientation of the collagen fibrils. In addition to influencing the effectiveness of the alignment, these similar parameters generally also affect the limit of alignment reached by the process. For example, if the collagen is initially more randomly oriented, the tissue can be artificially aligned to a greater degree. The selective alignment of the tissue results in measurable structural and property changes in the tissue in comparison with the corresponding tissue that is not selectively aligned. In general, the alignment of the tissue results in an increase in rigidity of the tissue along the alignment direction such that the tissue bends less easily along axes perpendicular to the alignment direction in comparison with corresponding tissue without the alignment. While not wanting to be bound by theory, it is thought that alignment of the tissue correlates with changes in orientation of collagen fibrils within the tissue. Specifically, alignment of the tissue properties is thought to correspond with collagen fibrils that are more aligned with each other than in tissue that has not been artificially aligned.

Collagen is a structural protein with an amino acid composition having a high glycine, proline and hydroxyproline content. Collagen forms molecules of a triple right-hand twisted helix of left-handed single chain protein helixes. Individual triple helix molecules are approximately 3000 angstroms (300 nanometers) long. The triple helices pack in a specific structure to form fibrils having a banded appearance from a staggered arrangement in the stacking. The fibrils are rigid and relatively inextensible. Connective tissue generally has a very high collagen content that accounts for the rigid nature of these tissues. The alignment of the collagen fibrils within natural tissue varies between different tissues. It is thought that alignment of the tissue through the application of a directional load alters the natural distribution of collagen alignment. The degree of alignment of the fibrils due to the application of a load may depend on the initial alignment of the collagen fibrils within the natural tissue.

Appropriate bioprosthetic tissue materials can be formed from natural tissues, synthetic tissue matrices and combinations thereof. Synthetic tissue matrices can be formed from extracellular matrix proteins that are combined to form a tissue matrix. Suitable tissues can comprise components of synthetic materials, such as polymers, that have or have had viable cells associated with the synthetic materials, in which the viable cells, when present, formed a proteinaceous extracellular matrix in association with any synthetic materials. Thus, tissue materials generally can have viable cells or protein materials formed from cells that are no longer present, whether or not synthetic materials are present. Suitable polymers, such as polyesters, and extracellular matrix proteins, such as collagen, gelatin, elastin, glycoproteins, silk collagen/elastin and combinations thereof, for incorporation into a synthetic tissue matrix are commercially available.

Natural, i.e. biological, tissue material suitable for alignment includes relatively intact tissue as well as decellularized tissue. Decellularized tissue can be obtained using chemical and/or biological agents, such as hypotonic buffers, hypertonic buffers, surfactants, proteases, nucleases, lipases, other similar agents and combinations thereof, to remove or dismantle cells and cellular structures within the extracellular matrix. These natural tissues generally include collagen-containing material. In particular, natural tissues may be obtained from, for example, native heart valves, portions of native heart valves such as roots, walls and leaflets, pericardial tissues such as pericardial sacs, amniotic sacs, connective tissues, bypass grafts, tendons, ligaments, skin patches, blood vessels, cartilage, dura mater, skin, bone, fascia, submucosa, such as intestinal submucosa, umbilical tissues, and the like. Natural tissues are derived from a particular animal species, typically mammalian, such as human, bovine, equine, ovine, porcine, seal or kangaroo. These tissues may include a whole organ, a portion of an organ or structural tissue components.

Suitable natural tissues include xenografts (i.e., cross species, such as a non-human donor for a human recipient), homografts (i.e., interspecies with a donor of the same species as the recipient) and autografts (i.e., the donor and the recipient being the same individual). Suitable tissue is typically, but not necessarily, soft tissue. While in principle any collagenous tissue can be oriented/aligned, as described herein, effects of alignment may be more pronounced in soft tissues. More rigid natural tissues may have a high collagen concentration and/or natural crosslinking of tissue that may inhibit artificial orientation of the tissue.

Synthetic tissue matrices can be formed from structural proteins, e.g., extra-cellular matrix components, that are assembled into tissue structures, such as sheets or other shapes. For example, purified collagen can be formed into a sheet of randomly oriented collagen. While purified collagen fibrils may be fragments of native collagen fibrils, orientation of the resulting tissues can be performed. Other materials, such as other structural proteins, can be combined with the collagen. Other structural proteins for incorporation into synthetic tissue matrices include, for example, elastin, proteoglycans and other glycoproteins. These other structural proteins can modify the properties of the tissue, for example, by introducing added flexibility and/or providing the material with a lower friction surface.

Several layers of tissue can be combined to form a fused tissue structure, which can have improved and/or more uniform properties. These combined layers can comprise fused layers of natural tissue, fused layer of synthetic tissue material or a combination of natural tissue layers and synthetic tissue layers. In particular, for the formation of tissue components for a heart valve, it may be advantageous to form a composite with one or more layers with a high collagen content combined with one or more layers with significant levels of elastin and/or proteoglycans. Natural and/or synthetic tissue layers can be fused together, for example, with lyophilization, adhesives, pressure and/or heat. Chemical crosslinking can also be used to fuse tissue layers together, although chemical crosslinking can inhibit alignment of the tissue. Natural or synthetic tissue matrices within a multiple layer structure can be aligned as individual layers before joining the layer or as the combined structure after joining and, optionally, fusing the layers. The use of pressure and/or heat to fuse intestinal submucosa tissue layers is described further in U.S. Pat. No. 5,955,110 to Patel et al., entitled “Multilayered Submucosal Graft Constructs And Methods For Making The Same,” incorporated herein by reference.

As a specific example of forming composites with a natural tissue and synthetic tissue matrices, intestinal submucosa can be combined with a synthetic layer comprising collagen, elastin and/or proteoglycans. Intestinal submucosa is a good source of uniform natural tissue with a high collagen content. However, intestinal submucosa alone is too rigid for some applications, such as for heart valve leaflets. In addition, intestinal submucosa is thin, such that it would be desirable to combine intestinal submucosa with additional layers, either other layers of natural tissue and/or synthetic materials. Thus, the combination of intestinal submucosa with synthetic layers including compositions that impart added flexibility can result in a composite material that has appropriate overall properties. Specific examples of composites formed with natural tissue, such as intestinal submucosa, and synthetic layers or a plurality of synthetic layers are described further in copending and commonly assigned U.S. patent application Ser. No. 10/027,464 to Kelly et al., entitled “Matrices For Synthetic Tissue,” incorporated herein by reference.

Tissue materials can be fixed by crosslinking. Fixation provides mechanical stabilization, for example, by preventing enzymatic degradation of the tissue and by anchoring the collagen fibrils. Glutaraldehyde, formaldehyde or a combination thereof is typically used for fixation, but other fixatives can be used, such as epoxides, epoxyamines, diimides and other difunctional aldehydes. In particular, aldehyde functional groups are highly reactive with amine groups in proteins, such as collagen. Epoxyamines are molecules that generally include both an amine moiety (e.g. a primary, secondary, tertiary, or quaternary amine) and an epoxide moiety. The epoxyamine compound can be a monoepoxyamine compound and/or a polyepoxyamine compound. In some embodiments, the epoxyamine compound is a polyepoxyamine compound having at least two epoxide moieties and possibly three or more epoxide moieties. In some embodiments, the polyepoxyamine compound is triglycidylamine (TGA). The use of epoxyamines as crosslinking agents is described further in U.S. Pat. No. 6,391,538 to Vyavahare et al., entitled “Stabilization Of Implantable Bioprosthetic Tissue,” incorporated herein by reference.

In general, the process to form fully crosslinked tissue requires a significant amount of time, in part, because the crosslinking agent must penetrate through the tissue. Also, the crosslinking process generally reaches a point of completion at which the properties of the tissue are relatively stabile with respect to any additional measurable changes upon further contact with the crosslinking agent. At the point of completion, it is thought that the crosslinking composition forms a stable crosslinked network. Presumably, at completion, many, if not all, of the tissue's available functional groups for crosslinking have reacted with a crosslinking agent. Since the formation of a fully crosslinked tissue is a slow process, the degree of crosslinking of the tissue can be selected to range from very low levels to completion of crosslinking. Upon completion of the collagen crosslinking, the crosslinked network generally fixes the relative orientation of collagen fibrils. However, the collagen of partially crosslinked tissue may be mobile enough to provide for alignment of the tissue during the crosslinking process, and some partial crosslinking of the tissue can be performed prior to performing the tissue alignment without preventing all changes in properties resulting from the alignment of the tissue.

The rigid collagen fibrils can be interwoven within the matrix of a tissue. The properties of a particular tissue depend on the overall composition of the tissue and any orientation of the collagen fibrils. A section of tissue will have various properties relevant to tissue function including, for example, flexibility with respect to deformation in response to shear or out-of-plane bending generally as well as extensibility with respect to elasticity and in plane expansion and/or compression. With respect to tissue composition, some tissues have elastin and/or proteoglycans that increase the elasticity and flexibility of the tissue.

Due to the rigidity of the collagen fibrils, orientation of the collagen fibrils can affect the overall rigidity of the tissue. For example, if the collagen fibrils are randomly oriented, the tissue will be more flexible than similar tissue with partially aligned fibrils. More specifically, if the fibrils are partially aligned in one direction, the tissue will be less flexible perpendicular to the direction of orientation of the fibrils. In other words, the rigidity of the collagen fibrils translates into rigidity of the tissue. Specifically, the bending of the tissue requires more force around axes that intersect, at angles closer to perpendicular, a line indicating the net average alignment of the fibrils. If the collagen fibrils of the tissue are aligned in more than one direction, the tissue can become more rigid perpendicular to the plurality of direction of collagen alignment. Thus, a cross-hatched array of collagen fibrils results in a more rigid matrix than a corresponding tissue with a corresponding random array of collagen fibrils. Two directions of tissue alignment may or may not be orthogonal.

Since any natural alignment of collagen fibrils can influence tissue properties, the protein structure of native tissue influences the physical properties of the native tissue. In addition, selective artificial alignment of tissue can alter the flexibility/rigidity of the tissue relative to the tissue prior to alignment.

Tissue sections of particular interest for forming heart valve prostheses generally have a thickness of at least about 50 microns, generally from about 75 microns to about 3 millimeters (mm) and in other embodiments from about 100 microns to about one (1) millimeter. A person of ordinary skill in the art will recognize that additional ranges of thickness within these explicit ranges are contemplated and are within the present disclosure. While tissue sheets can be effectively used to form various components, including, for example, both flat and curved components, tissue sections that are inherently non-planar are contemplated and can be process for tissue alignment, as described herein.

A particular approach can be used for evaluating flexibility of sheets of tissue that generally have a thickness from about 100 microns to about one (1) mm. The rigidity/flexibility of the tissue can be appraised by examining the bending of the tissue over a pivot. The tissue sheet can be cut into a disk with a diameter of about 1.75 inches (44.45 mm). The disk of tissue then is placed on a horizontal rod with a diameter of 0.2-0.3 inches (5.08 mm-7.62 mm) with the center of the disk approximately on the top of the rod. The amount of bending of the disk can be quantified with ratings of 0-3, as described in the examples below. Additionally or alternatively, one can consider the magnitude of tissue flexibility by a change in the angle at which the tissue drapes over the rod. In some embodiments, the tissue can hang vertically down over the rod. If the tissue is sticky, opposite edges of the tissue can stick together when approximately hanging vertically down. Alignment of the tissue results in a stiffer tissue that does not hang as low relative to the horizontal relative to an equivalent tissue without application of a load. For example, the tissue can hang over the rod at least about 10 degrees closer to the horizontal, in other embodiment at least about 20 degrees closer to the horizontal, in further embodiments at least about 40 degrees closer to the horizontal and in additional embodiments at least about 60 degrees closer to horizontal relative to an equivalent tissue without application of a load. A person of ordinary skill in the art will recognize that additional values within the explicit values of tissue hang angles are contemplated and are within the present disclosure.

The extensibility, i.e., the ability to extend or stretch, of the tissue generally is also effected by the alignment of tissue properties. The extensibility of native tissue depends on the degree of alignment of the fibers and the morphology of the tissue and can be variable between similar samples. By aligning the tissue as described herein, the variability of the extensibility can be reduced. More consistent in-plane extensibility can result in more consistent coaptation between the leaflets. For example, stented valves can have consistently proper coaptation of the leaflets if the extensibility is predictable since the leaflets can be cut appropriately.

Axial extensibility can be evaluated as the maximum stretch ratio under peak equibiaxial membrane stress of 60 Newtons/meter (N/m). The overall or net extensibility of a tissue element is given by the areal strain under 60 N/m tension, computed as (λ_{R}·λ_{C}−1)·100%, where λ_{R} and λ_{C} are radial and circumferential stretch ratios, respectively. The value of 60 N/m is selected to reasonably represent the deformation under peak diastolic load. In addition, the mechanical strength can be more consistent and aligned in a desirable manner following alignment of the tissue properties.

Tensioning Apparatuses for Orienting Collagen Fibrils in Tissue

A tensioning apparatus for performing the alignment of tissue applies a selected load to the tissue for an appropriate period of time to effect the alignment of the tissue with corresponding changes in tissue properties. To stress the tissue, a tensioning device transfers the force/load through suitable grippers to the tissue. A tensioning device can be stationary with the tissue stretched under tension and anchored in the tensed state. In other embodiments, the tensioning device applies a selected load to the tissue using a tensioning apparatus that can apply a selectable degree of load along one or more gripped but un-anchored edges of the tissue. The apparatus generally maintains the tissue in a moist condition to prevent irreversible modification of the tissue if the tissue dehydrates.

A representative tensioning apparatus is shown schematically in FIG. 5. Tensioning apparatus 300 can comprise an optional container/enclosure 302 that supports a tissue to be processed, a tensioning device 304, a moisture source 306 and an optional tissue support 308. Optional container/enclosure 302 can be used to immerse the tissue in liquid and/or to enclose the tissue in a moist environment. For example, container/enclosure 302 can be a tray, pan, tub, vessel or the like with sufficient liquid to cover the tissue. A container/enclosure 302 that immerses the tissue can be particularly desirable in embodiments in which the tissue is crosslinked during the tensioning process.

However, the tissue does not have to be immersed continuously to maintain the tissue sufficiently moist. Thus, container/enclosure 302 can be an enclosure covering the tissue to maintain a high moisture environment such that the tissue does not dry out. If container/enclosure 302 seals the tissue from the ambient environment, container/enclosure 302 generally has a liquid reservoir or the like in fluid communication with the atmosphere surrounding the tissue such that the humidity level within container/enclosure 302 is high or saturated, e.g., 100% relative humidity. This high humidity level prevents dehydration of the tissue. In additional embodiments, tissue can be suspended without a vessel/enclosure 302 if sufficient moisture is supplied to the tissue by moisture source 306 or if the humidity in the room is sufficiently high and the processing time is sufficiently short such that the tissue does not have time to dry out.

In a first embodiment, tensioning device 304 holds the tissue against a stationary frame. Referring to FIG. 6, device 310 comprises a frame 312 with spikes 314. Tissue 316 is stuck onto spikes 314. If tissue 316 is stretched onto spikes 314 under appropriate tension, the tissue is under tension on the tensioning device. This tension on the fastened tissue can be sufficient to align the tissue. The tissue can be stretched to a taught configuration in one or both orthogonal directions when fastening to the frame. In an alternative embodiment with a tissue held in place, the tissue is sutured to a stationary frame with the suture tied off under tension to apply a desired amount of tension. The suture can be applied to the appropriate edges to hold the tissue in place and to apply a desired amount of tension.

Similarly, if the tissue is applied to the frame without tension, the tissue can be treated while attached to the frame to shrink such that the shrinkage of the tissue results in a load on the tissue. For example, crosslinking tissue results in shrinkage of the tissue. If the shrinking is fast enough relative to the mechanical stabilization of the tissue, the load can align the tissue properties before the crosslinking fixes the properties of the tissue from further modification. The alignment of the tissue varies in time due to the process itself. Additionally or alternatively, the tissue on the frame can be indented with a shaped/contoured object, such as a cononical tip, to apply a load that spreads to adjacent tissue. In other words, a load is applied by thrusting an object against the bound tissue at a particular location. If the edges of the tissue are fixed, shrinkage of the tissue increases the load from the object over time.

In other embodiments, tensioning device 304 comprises gripper(s) 318, connector(s) 320, one or more load applicators 322 and, optionally a tension or load meter 324, as shown schematically in FIG. 5. Connector(s) 320 connect gripper(s) 318 with load applicator(s) 322 or to an anchor 326, as shown in FIG. 7. Anchor 326 is a stationary object that does not significantly move or deflect in response to forces applied through a connector 320. In some embodiments, tension in the tissue can be applied through the use of a load applicator 322 connected to one side of a tissue element while the opposite side of the tissue element is connected to an anchor. Thus, the use of an anchor 326 is an alternative to the use of two load applicators attached to opposing sides of tissue.

Gripper 318 can be suture, chord, wire, clamps, similar grippers and combinations thereof. In some embodiments, gripper 318 is a loop or other portion of connector 320, in which connector 320 comprises a suture, chord, wire or the like. Thus, a portion of connector 320 is stitched through the tissue to secure the tissue to connector 320. Gripper 318 can also be suture, chord, wire or the like even if connector 320 is formed from a different element as long as gripper 318 is appropriately fastened to connector 320. In addition, gripper 318 can be a clamp or the like either with a spring loading to grip the tissue, a screw down component or locking system to hold the clamp in a grip on the tissue. For example, conventional clamps can be used. The size of the clamp can be selected to distribute the load along the edge of the tissue. Similarly, a plurality of grippers 318, either suture stitches, clamps or the like, can be used along an edge of the tissue to distribute the load. The grippers may or may not alter or damage the tissue in contact with the gripper. In embodiments in which the tissue is subsequently trimmed following processing to orient the collagen, the portion of the tissue altered or damaged at the grippers, if any, may be removed during the trimming process.

Connectors 320 can be suture, wire, string, a rod, other like structures suitable for transmitting forces, or combinations thereof. In particular, in some embodiments, a plurality of connectors 320 and corresponding grippers 318 is used on each side of a tissue element to more evenly distribute forces along the tissue. A plurality of connectors 330 on each side of a tissue element can be combined into a single connector 332, which is attached to a load applicator 322, as shown in FIG. 8. The plurality of connectors 330 can be combined into single connector 332 by weaving, welding or other appropriate fastening approach, which is generally influenced by the material used to form connectors 330, 332. In alternative or additional embodiments, a plurality of connectors 334 connected to grippers on a single side of a tissue can go to separate load applicators 336, as shown in FIG. 9.

Load applicator 322 can comprise, for example, a weight, a motor, a spring or the like. In one embodiment, as shown in FIG. 10, a flexible connector 344 passes over pulley 346 to a weight 348. The amount of weight can be selected to provide a desired amount of load to tissue. Rather than using a pulley, the load applicator can be configured with the tissue hung vertically such that the tissue is under tension between a weight and an anchor. Such a configuration is shown in FIG. 11. A shown in FIG. 11, tissue 350 is attached to anchor 352 and clamp 354. Clamp 354 is connected to weight 356 with a connector 358. The tissue and weight can be suspended in a liquid. In the weight based embodiments, once a particular weight is selected, a reproducible amount of load can be applied to tissues by using the selected weight.

In alternative embodiments, load applicator 322 (FIG. 5) comprises a motor 360, as shown in FIG. 12. The motor has a suitable clutch such that the motor does not overheat with an immovable force provided by connector 320. For example, a stepper motor can be used to apply a selected displacement on connector 320 to apply a desired load. Also, an Instron Brand tensile tester (Instron Inc., Canton, Mass.) can be adapted for this use. The motor can apply a continuous load or a load that varies in a selected way with time. For example, the load can be periodically pulsed. Suitable frequencies for pulsing the tissue with a force range from about 0.1 hertz (Hz) to about 10 Hz and in other embodiments from about 0.4 Hz to about to about 4 Hz. At high enough frequencies, the tissue does not relax during the cycle and the effect of the pulsing may be lost. The application of a pulsed load may be effective in increasing the stiffness of the tissue without correspondingly decreasing the extensibility due to relaxation of the tissue during the relaxation portion of the cycle. The Instron can be used to apply either a pulsed or constant load. Again, the tissue can be oriented in a vertical or horizontal direction with one end of the tissue connected to an anchor and the other end is connected to the Instron instrument. In another embodiment for application of a load throughout the tissue in a radial direction, a tissue element 362 is attached to the top of a hollow cylinder 364, as shown in FIG. 13. A load can be applied with a deflection probe 366. Application of a radial load results in stress rings within the tissue.

Many different embodiments for applying tension can be based on springs and the like. In some embodiments, the load applied by the spring can be adjusted by turning a knob, lever or the like. A representative example is shown in FIG. 14. Connector 370 connects to spring 372. Spring 372 is connected to platform 374. Screw 376 connects to platform 374 through pivot 378 that freely rotates such that screw 376 can be rotated without rotating platform 374. Screw 376 connects through a threaded hole in mount 380. Knob 382 is connected to screw 376 such that rotation of knob 382 rotates screw 376. Thus, the position of platform 374 relative to mount 380 can be changed by rotation of knob 382 since the rotation of screw 376 moves screw 376 relative to mount 380. Altering the position of platform 374 changes the tension on spring 372 since the position of connector 370 generally is held approximately in place.

Moisture source 306 can be any supply of moisture that can be applied to the tissue. For example, moisture source 306 can be a liquid reservoir, a spray apparatus, a supply of humidity, combinations thereof, or the like. A liquid reservoir can be a liquid within container/enclosure 302. Referring to FIG. 15, liquid reservoir 384 is located within a tray 386, which can function as container/enclosure 302 (FIG. 5). The liquid can be buffered saline or other liquid that is compatible with the tissue. Suitable buffers and other compositions suitable for the liquid, such as a crosslinking agent, are described further below.

A spray apparatus can provide a continuous or intermittent flow of liquid to the tissue from one or a plurality of spray heads. If a plurality of spray heads are used, the spray heads can be connected to a common liquid reservoir, to separate liquid reservoirs or to a combination thereof, as desired. If separate liquid reservoirs are connected to separate spray heads, the liquid reservoirs can contain the same or different liquids. Referring to FIG. 16, spray apparatus 390 comprises two spray heads 392, 394 each of which is connected to a liquid reservoir 396. Liquid reservoir 396 contains suitable liquid for contacting tissue 398. The spray from spray heads 392, 394 is shown schematically in FIG. 16.

In alternative embodiments, moisture source 306 is a humidity source, which can be a liquid reservoir separate from the tissue. If the moisture source is a humidity source, container/enclosure 302 generally is an enclosure that seals or partially seals the tissue from the ambient atmosphere. The fluid of the humidity source can be a pool of liquid, which can be heated if desired to increase the vapor pressure, or it can be misted into the enclosure to ensure at least 100% relative humidity. Misting the liquid can also provide an aerosol within the chamber that supplies buffer and other compositions to the tissue in addition to water. Referring to FIG. 17, tissue 410, grippers 412, connectors 414, tensioning elements 416 and humidity source 418 are within enclosure 420 that seals off the ambient environment. Humidity source 418 includes a reservoir of liquid 422.

Referring to FIG. 5, optional tissue support 308 can provide a curved surface to shape or maintain the shape of the tissue during the application of a load to the tissue. For example, optional tissue support can provide a cylindrical surface or a hemisphere surface. The grippers 304 and corresponding connectors 306 are oriented to apply tension to the tissue positioned along the tissue support. Tissue support 308 is anchored such that tension on the tissue is maintained. In some embodiments with a tissue support, a single tensioning element can provide the appropriate tension on the tissue. For example, as shown in FIGS. 18 and 19, a hemisphere shaped tissue support 430 is connected to a mount 432. Tissue 434 is contoured along tissue support 430. A plurality of connectors 436 and corresponding gripper 438 extend around tissue 434. Plurality of connectors 436 joins to a single unified connector 440, which connects to tensioning apparatus 306 (FIG. 5). Moisture source 304 can be designed for appropriate use with a tissue support 308, for example, by having sufficient liquid to immerse the curved tissue element or by directing a spray appropriately.

Processing To Align Tissue

It has been discovered that application of an oriented load on a tissue, generally collagenous tissue, can result in alignment of the tissue. By crosslinking the tissue, the reorientation of the collagen fibrils is fixed in place. The conditions used for the processing of the tissue can influence the degree of alignment of the tissue that results in a stiffening of the tissue with respect to bending orthogonal to the force direction. Thus, the processing conditions can be selected to yield desired properties for the tissue based on intended application of the tissue. Stressing of tissue to align the tissue can lead to tissue with more uniform and reproducible properties as well as forming the tissue with desired rigidity in selected directions and flexibility with respect to other directions.

Appropriate apparatuses for applying a load to the tissue are described above. In general, the amount of force that is applied to the tissue ranges from about one (1) gram per centimeter (g/cm) to about 1000 g/cm, in further embodiments from about 5 g/cm to about 250 g/cm, and in other embodiments from about 10 g/cm to about 100 g/cm. A person of ordinary skill in the art will recognize that additional ranges within the explicit ranges of load are contemplated and are within the present disclosure. To induce selected or desired amounts of tissue alignment, the load generally is applied for about 1 minute to about 10 days, in other embodiments from about 10 minutes to about 10 days, and in further embodiments from about 1 hour to about 48 hours. A person of ordinary skill in the art will recognize that additional ranges of load magnitudes and times within the explicit ranges are contemplated and are within the present disclosure. As noted above, the load can be pulsed or cycled. By appropriate adjustment of the magnitude of load, the processing time and the processing conditions, the degree of alignment of the tissue can be selected over an available range of tissue properties. In addition to pulsing the load, the magnitude of the load, whether or not pulsed, can be varied during the processing. If the tissue is crosslinked while under a load, the modification of the tissue due to the stress will gradually stop due to crosslinking of the tissue. Therefore, once the tissue is fully crosslinked the tissue can be maintained under the load without further modifying the tissue properties.

In general, the moisture used for maintaining the tissue in a hydrated condition during the step of applying a load is sterile. If the tissue is immersed or sprayed with a liquid during the loading process, the liquid may or may not include a crosslinking agent. Suitable liquids include, for example, buffered saline or the like. In general, buffered saline can have an ionic strength similar to physiological liquids, such as blood, such that the tissue is not modified by the ionic strength of the liquid. Similarly, the liquid can have a pH near a physiological pH to avoid modifying the tissue due to pH. In particular, the liquid preferably is buffered at a near physiological pH ranging from about 6.0 to about 10.0, and in other embodiments ranging from about 6.9 to about 9.0. Suitable buffers can be based on, for example, the following compounds: ammonium, phosphate, borate, bicarbonate, carbonate, cacodylate, citrate, and other organic buffers such as tris(hydroxymethyl) aminomethane (TRIS), N-(2-hydroxyethyl)piperazine-N′-(2-ethanesulfonic acid) (HEPES), and morpholine propanesulphonic acid (MOPS).

The tissue is generally fully crosslinked either during, after or both during and after applying a load to the tissue. If the crosslinking is performed during the process of applying a load, the tissue is immersed or sprayed with a liquid solution comprising a crosslinking agent generally dissolved in an aqueous buffered saline solution. For glutaraldehyde crosslinking, the solution generally has a concentration of glutaraldehyde from about 0.001 weight percent to about 10 weight percent, in other embodiments, from about 0.05 weight percent to about 2 weight percent. For crosslinking with epoxyamines, the solution generally has a crosslinker concentration from about 0.001 molar (M) to about 2M and in other embodiments from about 0.01M to about 1M. A person of ordinary skill in the art will recognize that additional values of crosslinker concentrations within these explicit valves are contemplated and are within the present disclosure.

The crosslinking generally is performed for at least about 1 hour, in some embodiments from about 2 hours to about 360 hours and in further embodiments from about 4 hours to about 240 hours. With epoxyamine crosslinkers, the crosslinking generally is performed for at least about 1 hour, and generally from about 3 days to about 10 days. A person of ordinary skill in the art will recognize that additional values of crosslinking times within these explicit valves are contemplated and are within the present disclosure. Similarly, a plurality of crosslinking agent can be used either sequentially or simultaneously. In general, crosslinking is performed for at least about 24 hours to fully crosslink the tissue, although the amount of time required can depend on the solution and other conditions during the crosslinking process. Since crosslinking generally is performed to completion prior to use of the medical device, crosslinking can be continued indefinitely without altering the tissue properties from the properties of the fully crosslinked tissue. Specifically, as with aldehyde crosslinkers, the crosslinking with epoxyamines reaches a point of completion, and the crosslinked tissue can be stored in the epoxyamine solution following completion of the crosslinking. In particular, tissue can be stored in glutaraldehyde or other crosslinking agent as a sterilant. Also, as noted above, a light crosslinking can be performed prior to application of a load.

In general, the crosslinking can be performed with two increments in which the load is applied during the first increment. The crosslinking during the first increment while a load is applied is sufficient to fix the orientation of the tissue during the remaining crosslinking process. For example with glutaraldehyde crosslinking, the load can be applied for 2 to 48 hours of the crosslinking time to fix the tissue orientation. Then, the load is removed, and the crosslinking process is continued to completion, generally at least about another 96 hours.

If the crosslinking is performed following completion of the application of a load, it may be desirable to perform the crosslinking without storing the tissue for significant periods of time. In particular, the alignment of tissue properties may alter with the passage of time since the tissue is not crosslinked to fix the tissue properties. A small loss of alignment of tissue properties can be accounted for when evaluating the degree of alignment to be obtained. In general, it is desirable to crosslink the tissue immediately or shortly after completing the alignment to fix the alignment more predictably and reproducibly.

Other processing of the tissue can be performed simultaneously with the application of a load or after the application of a load. In particular, biological agents can be associated with the tissue to impart desired properties to the tissue. In particular, some of these desired biological agents can be associated with the tissue prior to, during or after crosslinking of the tissue. These biological agents are described further in the following section.

Additional Tissue Processing

Besides crosslinking, the tissue can be treated with other compounds to modify the tissue properties. Specifically, the tissue can be further modified, for example, to reduce calcification of the tissue following implantation and/or to encourage colonization of the tissue with desired cells. In particular, to encourage colonization with desired cells, the tissue can be treated to reduce or eliminate toxicity associated with aldehyde crosslinking and/or associated with compounds that stimulate the association of desirable cells with the tissue.

In some embodiments, tissue crosslinked with dialdehydes or the like can be treated to reduce or eliminate any cytotoxicity. Compositions for the treatment of aldehyde crosslinked tissue are described further in copending and commonly assigned U.S. patent application, Ser. No. 09/480,437 to Ashworth et al., entitled “Biocompatible Prosthetic Tissue,” incorporated herein by reference.

Generally, any calcification reducing agents would be contacted with the composite matrix following crosslinking. Suitable calcification reducing agents include detergents (e.g., sodium dodecyl sulfate), toluidine blue, diphosphonates, and multivalent cations, especially Al⁺³, Mg⁺² or Fe⁺³, or corresponding metals that can oxidize to form the multivalent metal cations. The effectiveness of AlCl₃ and FeCl₃ in reducing calcification of crosslinked tissue is described in U.S. Pat. No. 5,368,608 to Levy et al., entitled “Calcification-Resistant Materials and Methods of Making Same Through Use of Multivalent Cations,” incorporated herein by reference. The delivery of anti-calcification agents using microscopic storage structures is described in U.S. Pat. No. 6,193,749 to Schroeder et al., entitled “Calcification Resistant Biomaterials,” incorporated herein by reference.

For some natural tissues, including heart valves, the underlying native tissue includes fibroblast cells within an extracellular matrix. The fibroblast cells produce and maintain the extracellular matrix. The surface of a vascular/cardiovascular tissue has a layer of endothelial cells a few cells thick. The endothelial cells provide desirable surface properties to the tissue for blood flow. Specifically, the endothelial cells form a blood contacting surface that is highly non-thrombogenic and blood compatible. Additional treatment of artificially aligned tissue to stimulate association of desirable cells with the tissue with oriented collagen can involve affiliation of appropriate compounds, especially proteins, with the tissue.

For example, after processing of the tissue to align the tissue, the tissue can be associated with one or more growth factors, such as vascular endothelial growth factor (VEGF) and/or fibroblast growth factor, and/or compounds that attract cell precursors to the tissue, attraction compounds. Suitable colonization stimulating compounds can assist with cellular attachment or the compounds can stimulate cellular proliferation. The compounds are selected based on the desired cell types for colonization of the fixed aligned tissue to form a biosynthetic tissue. The use of growth factors, such as VEGF, in the production of prostheses has been described further in copending and commonly assigned U.S. patent applications Ser. No. 09/014,087 to Carlyle et al., entitled “Prostheses With Associated Growth Factors,” and Ser. No. 09/186,810 to Carlyle et al., entitled “Prostheses With Associated Growth Factors,” both of which are incorporated herein by reference. Fibroblast growth factors refer to a group of proteins that are characterized by the binding of heparin. These proteins have also been called heparin binding growth factors. These proteins strongly stimulate the proliferation of fibroblasts and possibly a variety of other cells of meodermal, ectodermal and endodermal origin.

The use of attraction compounds to associate precursor cells with a substrate is described further in copending and commonly assigned U.S. patent application Ser. No. 09/203,052 to Carlyle et al., entitled “Substrates For Forming Synthetic Tissue,” incorporated herein by reference. The association of a colonization stimulating composition, e.g., a growth factor and/or an attraction compound, with a tissue matrix may involve direct attachment, application of a coating, including an adhesive or binder, or chemical binding, involving a binding agent in addition to the attraction compound/response modifier.

Assembly Of Medical Devices

The selectively aligned tissue elements can be assembled, if necessary, into a variety of medical devices following the alignment of the tissue. In some embodiments, the tissue forms the entire medical device. In relevant embodiments involving assembly, if crosslinking is performed or completed following selective alignment of the tissue, the medical device can be assembled prior to crosslinking or other processing of the tissue, in some embodiments. While various prostheses, as described above, can be produced from the selectively aligned tissue, there is particular interest in fabricating tendons, valved prostheses and/or biological conduits. Valved prostheses formed from tissue have flexible leaflets extending across the lumen of the valve. A leaflet support structure provides the framework for the support of the leaflets.

The different components of a prosthesis can incorporate one or more tissue elements and, optionally, additional synthetic materials. If a plurality of tissue elements are included in the medical device, each tissue element may or may not be processed to selectively align the tissue. Similarly, different tissue elements within a medical device may be treated differently with respect to the selected degree of alignment. Since the aligned tissue generally has an orientation, the orientation of the tissue generally is taken into account when incorporating the tissue into the medical device, although if the tissue is aligned in multiple dimensions the material may become stiffer in general without reference to a particular axis.

While the tissue can be cut after processing to selectively align the tissue, the tissue in principle can be cut to a desired size and/or shape prior to selectively aligning the tissue. The cutting of the tissue can be performed before or after treatment with any biologically active compositions for modifying the tissue properties. Similarly, the assembly of the prosthesis components, if required, also can be performed before or after treatment of the tissue with any biologically active compositions.

As an example of the assembly process, the heart valve prosthesis of FIG. 1 can be assembled from three structures of tissue portions, as shown in FIGS. 20-22. While each tissue element can be formed from selectively aligned tissue, the element forming the leaflet is particularly suitable for formation from selectively aligned tissue. Referring to FIG. 20, three leaflet segments 450 are used to form valve 100 (FIG. 1). One leaflet segment 450 forms each of the leaflets 102, 104, 106 in the completed valve 100. Each leaflet segment 450 includes a rounded portion 452, ears 454 and a free edge 456 extending between ears 454.

The tissue segment generally would be oriented with tissue aligned along the arrow shown in FIG. 20. For example, the tissue can be aligned by pulling opposite edges in the directions shown by the arrow. The tissue then remains flexible around axes parallel with the arrow and stiff with respect to bending around axes perpendicular to the arrow. In other words, the leaflets have a greater rigidity with respect to bending around axes extending from the attached edge to the free edge of the leaflet relative to bending around axes perpendicular to lines extending from the attached edge to the free edge. Such an orientation of the tissue provides for reproducible mechanical performance of the leaflet while enhancing coaptation of the valve. In particular, the free edge of the leaflets become stiffer such that the edges coapt when the valve is closed without partial collapsing of the leaflet edges in response to the fluid pressures against the closed valve.

Referring to FIG. 21, post segments 108 include rectangular tissue segments 460 with a slit 462. Slit 462 is placed over two adjacent leaflets with ears 454 of the two leaflets joined at post segment 108. Once the three leaflets are attached with three post segments 108, free edges 456 of the leaflets extend between post segments 108. By attaching ears 454 to post segment 108, post segment 108 reinforces a commissure post of the valve.

Referring to FIG. 22, bias strip 116 includes curved scalloped sections 464, 466, 468 joined by post sections 470, 472, 474. Scalloped sections 464, 466, 468 are joined to the three respective rounded portions 452 of the three leaflets segments 450. Once joined to the leaflet segments 450, scalloped sections 464, 466, 468 form inflow edge 124 of the valve. Post sections 470, 472, 474 join with post segments 108 and ears 454. Thus, leaflet segments 450 are secured along all of their edges except for free edges 456. Ends 476, 478 of bias strip 116 are secured along a leaflet segment such that bias strip 116 is attached along the circumference of valve 100. Aortic valve prosthesis 100 can be implanted into a patient with a single suture line for faster implantation. The tissue sections can be attached, for example, with suture, adhesives, staples or the like.

Similarly, the four-leaflet heart valve prosthesis of FIG. 2 can be assembled from four tissue components that are joined together to form the valve. These components are shown in FIGS. 23-26. Referring to FIGS. 23-26, leaflet sections 500, 502, 504, 506 each have a section corresponding to one of leaflets 134, 136, 138, 140, respectively. Leaflet sections 500, 502, 504, 506 generally would be oriented similarly to leaflet segment 450 as indicated by the arrows in FIGS. 23-26. In other words, the leaflets are oriented to have greater flexibility with respect to bending around axes extending from the chordae to the free edge of the leaflet relative to bending around axes perpendicular to axes extending from the chordae to the free edge. Leaflet sections 500, 502, 504, 506 further include edge sections 510, 512, 514, 516, respectively. Edge sections 510, 512, 514, 516 together form edge 146 that is secured to the sewing ring by insertion between portions 138 and 150 of sewing ring 132, as shown in FIG. 2.

Referring to FIGS. 23-26, folds 518 separate edge sections 510, 512, 514, 516 from leaflets 134, 136, 138, 140. Specifically, leaflets 134, 136, 138, 140 are formed between folds 518 and chordae 142. Slits 520 are cut in leaflet sections 502, 506 to form chordae 142. Similarly, slots 522 are cut in leaflet sections 500, 504 to form chordae 142. Attachment sections 144 extend from the bottom of chordae 142. Additional structures, such as tabs 524, can be included to facilitate assembly of the prosthesis.

To assemble the tissue components, leaflet sections 500, 502, 504, 506 are attached to adjacent leaflet sections. Generally, the tissue can be aligned along the arrows shown in FIGS. 23-26 to obtained desired leaflet function. In particular, orienting the tissue as shown with the arrows improves leaflet function by decreasing flexibility of the leaflet between the bottom of the chordae and the valve annulus during use. Attachment sections 144 are secured into two groupings with one of the two attachment sections 144 of leaflet sections 502, 506 being attached to each group. Chordae 142 remain unattached to decrease interference with blood flow. Edge sections 510, 512, 514, 516 are attached to a sewing ring, as shown in FIG. 2. Attachment can be performed with suture or other attachment approaches. Assembly of a similar valve prosthesis is described U.S. Pat. No. 5,415,667 to Frater, entitled “Mitral Heart Valve Replacement,” incorporated herein by reference.

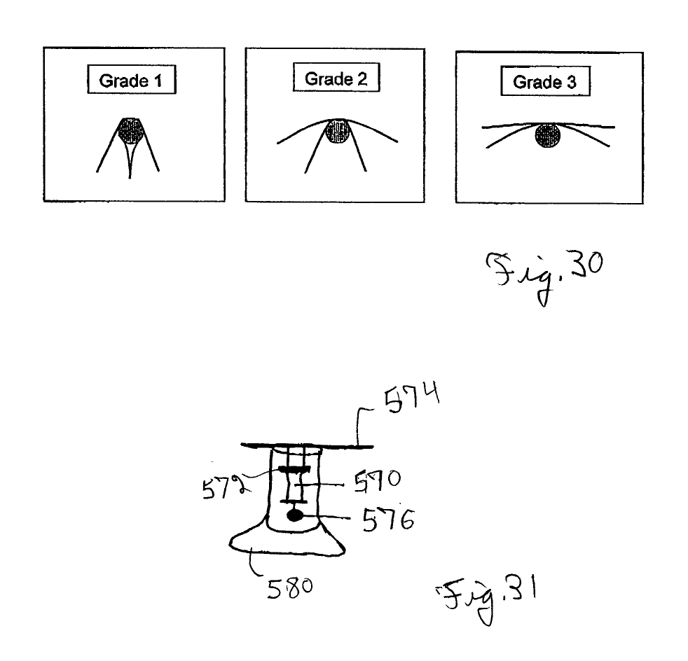

The stented valve of FIG. 3 can be assembled from a stent 162 and three tissue segments, with one segment for each leaflet. Stent 162 and one tissue segment 540 are shown in FIG. 27. Stent 162 has three commissure posts 542 and three scallops 544 between the commissure posts that together form a band 546 at the inflow edge 548. Referring to FIG. 28, a tissue segment 540 can be initially sutured, stapled, secured with an adhesive or otherwise fastened along the lower edge of the tissue segment toward the inflow edge 548 of the valve. As shown in FIG. 28, suture line 550 is stitched with a curved suture needle 552. After two adjacent tissue segments are secured, a suture line or other fastening approach can be used to secure the tissue segments along a commissure post 542. Referring to FIG. 29, a suture line 554 is shown partially formed along a commissure post. As shown in FIGS. 27-29, tissue segments 540 are contoured, although planar tissue segments can be similarly attached to stent 162, and the structure of stent 162 conforms a flat tissue segments to the desired leaflet shape upon application of the corresponding fluid pressures.

With respect to the biological conduit shown in FIG. 4A, in some embodiments, it is desirable for the conduit not to dilate, i.e., expand in diameter, following implantation. Thus, in these embodiments, the tissue is placed in the cylindrical configuration with the tissue aligned as indicated with the arrow. The stiffness introduced by the collagen alignment inhibits dilation of the conduit. This inhibition of dilation can be particularly useful for valved conduits, such as venous valved conduit or a section of the aorta near the aortic heart valve. Biological conduits for replacement of the aorta or the pulmonary artery are described further in copending and commonly assigned U.S. patent application Ser. No. 10/056,774 to Holmberg et al., entitled “Conduit For Aorta Or Pulmonary Artery,” incorporated herein by reference.

Along with tissue components, the medical devices can also comprise one or more other biocompatible materials, such as polymers, ceramics and metals. For example, stents and the like are generally formed from non-tissue materials. Appropriate ceramics include, without limitation, hydroxyapatite, alumina and pyrolytic carbon. Biocompatible metals include, for example, titanium, cobalt, stainless steel, nickel, iron alloys, cobalt alloys, such as Elgiloy®, a cobalt-chromium-nickel alloy, MP35N, a nickel-cobalt-chromium-molybdenum alloy, and Nitinol®, a nickel-titanium alloy.

Polymeric materials can be fabricated from synthetic polymers as well as purified biological polymers. Appropriate synthetic materials include hydrogels and other synthetic materials that cannot withstand severe dehydration. Suitable polymers include bioresorbable polymers that are gradually resorbed after implantation within a patient.

Appropriate synthetic polymers include, without limitation, polyamides (e.g., nylon), polyesters, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, ethylene vinyl acetates, polysulfones, nitrocelluloses and similar copolymers. Bioresorbable synthetic polymers can also be used such as dextran, hydroxyethyl starch, derivatives of gelatin, polyvinylpyrrolidone, polyvinyl alcohol, poly[N-(2-hydroxypropyl) methacrylamide], poly(hydroxy acids), poly(epsilon-caprolactone), polylactic acid, polyglycolic acid, poly(dimethyl glycolic acid), poly(hydroxy butyrate), and similar copolymers. These synthetic polymeric materials can be formed into fibers or yarns and then can be woven or knitted into a mesh to form a matrix or substrate. Alternatively, the synthetic polymer materials can be extruded, molded or cast into appropriate forms.

Biological polymers can be naturally occurring or produced in vitro by fermentation and the like or by recombinant genetic engineering. Purified biological polymers can be appropriately formed into a substrate by techniques such as weaving, knitting, casting, molding, extrusion, cellular alignment and magnetic alignment. Suitable biological polymers include, without limitation, collagen, elastin, silk, keratin, gelatin, polyamino acids, polysaccharides (e.g., cellulose and starch) and copolymers thereof.

Colonization of the Tissue With Cells

Some embodiments of the aligned tissue are suitable for in vivo or in vitro affiliation of cells with the tissue, although the tissue can be useful in some applications even if no cell colonization takes place. For in vivo affiliation with cells following implantation, the aligned tissue is assembled into a desired medical device and implanted. If the aligned tissue is prepared for cell colonization, the tissue is suitable seeding ground for cell colonization by cells that are circulating in the patient's fluids. Thus, circulating cells of the patient affiliate with the tissue and can form a repopulated biosynthetic tissue material.

In vitro cell colonization is performed in a cell culture system. With in vitro colonization, the cell colonization can be performed prior to or after assembly of the aligned tissue into a medical device. In some embodiments, a combination of in vivo and in vitro cell colonization can be used. For example, inner layers of the tissue can be colonized by selected cells in vitro to provide cell proliferation within the tissue while additional cell types can be colonized in vivo.

The in vitro affiliation of cells with the aligned tissue involves placing the aligned tissue into a cell culture system with the desired cells. The cell culture system can include one or more different cell types. Alternatively, the aligned tissue can be transferred sequentially to different cell culture systems, each with one or more cell types, for the association of the tissue with multiple cell types. To reduce the possibility of transplant rejection, the mammalian cells used for in vitro colonization preferably are autologous cells, i.e., cells from the ultimate recipient. In vitro affiliation of cells with tissue can be performed at hospitals where the patient's cells can be removed for use in a cell culture system. Appropriate cells include, for example, endothelial cells, fibroblast cells, corresponding precursor cells and combinations thereof. Association of endothelial cells is particularly appropriate in the production of prostheses that replace structures that naturally have an endothelial or epithelial cell lining, such as vascular components, cardiovascular structures, portions of the lymphatic system, uterine tissue or retinal tissue. Fibroblasts are capable of a variety of different functions depending on their association with a specific tissue. Myofibroblasts are fibroblasts that express relatively more contractile proteins such as myosin and actin.

The cells can be harvested from the patient's blood or bone marrow. Alternatively, suitable cells could be harvested from, for example, adipose tissue of the patient. The harvesting process can involve liposuction followed by collagenase digestion and purification of microvascular endothelial cells. A suitable process is described further in S. K. Williams, “Endothelial Cell Transplantation,” Cell Transplantation 4:401-410 (1995), incorporated herein by reference and in U.S. Pat. Nos. 4,883,755, 5,372,945 and 5,628,781, all three incorporated herein by reference.

Purified endothelial cells can be suspended in an appropriate growth media such as M199E (e.g., Sigma Cell Culture, St. Louis, Mo.) with the addition of autologous serum. Other cell types can be suspended similarly. The harvested cells can be contacted with the aligned tissue in a cell culture system to associate the cells with the tissue. Thus, a biosynthetic tissue is formed based on cells from the patient prior to implantation.

An aligned tissue can be incubated in a stirred cell suspension for a period of hours to days to allow for cell seeding. Cell seeding provides random attachment of cells that can proliferate to line the surface of the prosthetic substrate either before or after implantation into the patient. Alternatively, the aligned tissue can be incubated under a pressure gradient for a period of minutes to promote cell sodding. A suitable method for cell sodding can be adapted from a procedure described for vascular grafts in the S. K. Williams article, supra.

In addition, the aligned tissue can be placed in a culture system where the patient's cells, such as endothelial cells, are allowed to migrate onto the surface of the prosthetic substrate from adjacent tissue culture surfaces. If either attachment or migration of endothelial cells is performed under conditions involving physiological shear stress, then the endothelial cells colonizing the surface of the aligned tissue may express appropriate adhesion proteins that allow the cells to adhere more tenaciously following implantation.

Storage And Use Of Tissue And Tissue-Based Devices

The selectively aligned tissue can be stored prior to or after formation into a prosthesis, if relevant. Suitable storage techniques generally have a low risk of microbial contamination. For example, the tissue can be stored in a sealed container with sterile buffer, saline solution and/or an antimicrobial agent, such as glutaraldehyde or alcohol.

For distribution, the selectively aligned tissue generally is assembled into a prosthesis. The prostheses can be placed in sealed and sterile containers for shipping. To ensure maintenance of acceptable levels of sterility, the tissue can be transferred to the sterile container using accepted aseptic protocols. The containers can be dated such that the date reflects an appropriate advisable storage time.

The containers generally are packaged with instructions for the use of the medical devices along with desired and/or required labels. The containers are distributed to health care professionals for surgical implantation of the medical device, e.g., prostheses. The implantation is performed by a qualified health care professional. The surgical implantation generally involves the replacement or supplementation of damaged tissue with the prosthesis.

### EXAMPLES

### Example 1

Alignment of Tissue with a Spiked Frame

This example demonstrates the ability to form artificially and selectively aligned tissue by applying the tissue to a stationary frame.

Bovine pericardial tissue sections were obtained from a USDA approved abattoir. The tissue sections were cleaned of fat and sectioned into sheet elements of approximately 20 cm×20 cm. The sheets were stretched over a frame with spikes along four sides, as shown in FIG. 6. The tissue was pushed onto the spikes to hold the tissue in place. A total of sixty samples were divided into three groups of twenty samples each. The first group (Group A) was stretched taught in one direction and was relaxed in the orthogonal direction. The second group (Group B) was stretched taught in both directions. The third group (Group C) was relaxed in both directions.

The tissue segments were attached to the frame and submerged in a 0.5% buffered glutaraldehyde solution for 7 days. The glutaraldehyde solution was prepared by diluting 1:100 by volume a 50% by weight glutaraldehyde stock solution (EM Science, Cincinnati, Ohio) and included 55 mM HEPES buffered saline. After removing the tissue from the glutaraldehyde solution, the tissue was then cut into a disk using a 1.75 inch circular die.

The tissue samples were tested along the directions established by the frame, i.e., in the two orthogonal directions of the spikes. The tissue segments were placed on a rod with a diameter of 0.2 inches (5.08 mm) with the center of the disk on the rod. The tissue was draped over the rod at an angle related to the flexibility of the tissue. The degree of bending was graded according to the scale with ranges shown in FIG. 30. The results are shown in Table 1.

TABLE 1

| | Group A | | Group B | | Group C | |
|---|---|---|---|---|---|---|
| | Dominant | Cross | Dominant | Cross | Dominant | |
| | Fiber | Fiber | Fiber | Fiber | Fiber | Cross |
| # Sample | Direction | Direction | Direction | Direction | Direction | Fiber Direction |
| 1 | 1 | 3 | 3 | 3 | 1 | 1 |
| 2 | 1 | 3 | 2 | 3 | 1 | 1 |
| 3 | 1 | 3 | 3 | 3 | 1 | 2 |
| 4 | 1 | 3 | 3 | 3 | 1 | 2 |
| 5 | 1 | 3 | 3 | 3 | 1 | 1 |
| 6 | 2 | 3 | 2 | 3 | 1 | 1 |
| 7 | 1 | 3 | 3 | 3 | 1 | 2 |
| 8 | 1 | 3 | 2 | 3 | 1 | 2 |
| 9 | 1 | 2 | 2 | 2 | 1 | 1 |
| 10 | 1 | 3 | 3 | 3 | 1 | 1 |
| 11 | 1 | 3 | 3 | 3 | 1 | 1 |
| 12 | 1 | 3 | 2 | 2 | 1 | 1 |
| 13 | 2 | 3 | 3 | 3 | 2 | 2 |
| 14 | 1 | 3 | 3 | 3 | 1 | 1 |
| 15 | 1 | 3 | 3 | 3 | 1 | 1 |
| 16 | 1 | 3 | 3 | 3 | 1 | 2 |
| 17 | 1 | 3 | 3 | 3 | 1 | 1 |
| 18 | 1 | 3 | 3 | 3 | 1 | 1 |
| 19 | 1 | 3 | 2 | 3 | 1 | 1 |
| 20 | 1 | 3 | 3 | 3 | 1 | 1 |
| Average | 1.10 | 2.95 | 2.70 | 2.90 | 1.05 | 1.30 |
| SD | 0.31 | 0.22 | 0.47 | 0.31 | 0.22 | 0.47 |

The results in Table 1 indicate clearly that load applied by stretching the tissue under tension onto a frame can successfully align the tissue properties to stiffen the tissue with respect to bending along one or more dimensions. The tissue was significantly stiffer perpendicular to the load direction than perpendicular to directions that were not under a load. In particular, the bending stiffness increased around axes perpendicular to vectors aligned with the applied load, as desired and expected. Thus, the bending properties can be selectively altered through the application of a load.

In addition, the tear strength of the tissue was evaluated using a suture retention test. Samples from Group A were able to sustain loads more than 15 Newtons while retaining the suture of approximately 1.25 times greater than the loads sustained while retaining the suture for corresponding samples from Group C.

### Example 2

Aligning Tissue with a Weight

This example demonstrates that a weight can be used to align a tissue. The use of a weight provides a more quantifiable approach to application of a load then directly available using the frame of Example 1.

Bovine pericardial tissue segments were obtained from a USDA approved abattoir. The tissue segments were cleaned of fat and sectioned into strips approximately 2.5 cm×5 cm. As shown in FIG. 31, one edge of the tissue 570 was attached to a clamp 572. Clamp 572 was suspended from a bar 574. A 100 gram weight 576 was attached to a clamp 578 attached to the opposite edge of the tissue. The tissue and weight were suspended in a vessel 580. Vessel 580 contained 0.5% buffered glutaraldehyde solution that was described further in Example 1. The tissue and weight were immersed in the glutaraldehyde solution for 7 days.

Three samples were tested. Each of the samples had the equivalent flexibility of a grade three orthogonal to the stretching direction and a grade one along the stretching direction. Thus, the use of the apparatus of FIG. 31 was effective to align the tissue.

The embodiments above are intended to be illustrative and not limiting. Additional embodiments are Within the claims. Although the present invention has been described with reference to preferred embodiments, workers skilled in the art will recognize that changes may be made in form and detail without departing from the spirit and scope of the invention.

What is claimed is:

1. A method for processing tissue, the method comprising applying a sufficient directional load to the tissue to increase the rigidity of the tissue asymmetrically relative to an unaligned tissue equivalently processed that is not subjected to the load.

2. The method of claim 1 wherein the tissue is generally planar.

3. The method of claim 1 wherein the tissue is curved.

4. The method of claim 1 wherein the directional load has a magnitude from about 1 gram/centimeter (g/cm) to about 1000 g/cm.

5. The method of claim 1 wherein the load is applied for at least about 10 minutes.

6. The method of claim 1 wherein the load is applied for from about 1 hours to about 48 hours.

7. The method of claim 1 wherein a round 1.75 inch (44.45 mm) diameter section of the tissue prior to applying the direction load drapes vertically down over a rod with a diameter of 0.2 inches (5.08 mm).

8. The method of claim 1 wherein a round 1.75 inch (44.45 mm) diameter section of the tissue after applying the directional load, when draped over a 0.2 inch (5.08 mm) diameter rod, hangs at least about 20 degrees closer to the horizontal relative to an equivalent section of the tissue without application of a load.

9. The method of claim 1 wherein a round 1.75 inch (44.45 mm) diameter section of the tissue after applying the direction load when draped over a 0.25 inch (6.35 mm) diameter rod hangs at least about 40 degrees closer to the horizontal relative to an equivalent section of the tissue without application of a load.

10. The method of claim 1 wherein the tissue is contacted with a crosslinking agent while the load is applied, thereby crosslinking the tissue simultaneously while increasing the rigidity of the tissue.

11. The method of claim 1 further comprising crosslinking the tissue following completion of applying the directional load to the tissue.

12. The method of claim 1 wherein the tissue is maintained in a hydrated state while applying the load.

13. The method of claim 1 wherein the tissue is immersed in a liquid while applying the load.

14. The method of claim 1 further comprising associating the tissue with a growth factor.

15. The method of claim 1 wherein the directional load is applied continuously.

16. The method of claim 1 wherein the directional load is applied with a periodic oscillation.

17. The method of claim 1 wherein the directional load is applied with a weight.

18. The method of claim 1 wherein the directional load is applied with a motor.

19. The method of claim 1 wherein the directional load is applied by anchoring the tissue under tension.

20. The method of claim 1 wherein the tissue is gripped with a clamp while applying the load.

21. A method for processing tissue, the method comprising applying a sufficient load to the tissue to increase the rigidity of the tissue relative to an unaligned tissue equivalently processed that is not subjected to the load, wherein a load applicator applies the load to the tissue and wherein a connector transfers load from the load applicator to the tissue.

22. A method for forming a prosthetic valve, the method comprising assembling a plurality of leaflets to form a valve, wherein the tissue leaflet comprises selectively aligned tissue having asymmetric mechanical properties.

23. The method of claim 22 wherein the leaflets are oriented within the valve to have improved coaptation relative to a structure with corresponding tissue leaflets that do not have aligned properties.

24. The method of claim 22 wherein assembling the plurality of leaflets comprises connecting a tissue leaflet to a leaflet support structure.

25. The method of claim 24 wherein the leaflet support structure comprises a stent and wherein the tissue leaflet is fastened to the stent along an attached edge.

26. The method of claim 25 wherein the leaflet is oriented to have greater rigidity with respect to bending around axes extending from the attached edge to the free edge of the leaflet relative to bending around axes perpendicular to axes extending from the attached edge to the free edge.

27. The method of claim 22 wherein the leaflets are attached to chordae.

28. The method of claim 27 wherein the leaflet is oriented to have a greater flexibility with respect to bending around axes extending from the chordae to the free edge of the leaflet relative to bending around axes perpendicular to axes extending from the chordae to the free edge.

29. The method of claim 22 wherein assembling the plurality of leaflets comprises connecting a tissue leaflet to a leaflet support structure and wherein the connecting of the tissue to the leaflet support structure comprises suturing the tissue to the leaflet support structure.

30. The method of claim 22 further comprising cutting the tissue leaflet from a larger section of tissue.

31. The method of claim 30 wherein the larger section of tissue is generally planar.

32. The method of claim 30 wherein the larger section of tissue is curved.

33. The method of claim 22 wherein the prosthesis comprises a plurality of leaflets and the method further comprises connecting additional leaflets to the leaflet support structure to form the prosthesis with the plurality of leaflets.

34. Biocompatible tissue comprising selectively aligned tissue having an asymmetric flexibility, wherein the tissue comprises pericardial tissue, amniotic sac tissue, blood vessel tissue, cartilage, dura mater tissue, skin tissue, fascia tissue, submucosa tissue, or umbilical tissue.

35. The biocompatible tissue of claim 34 wherein the tissue is not crosslinked.

36. The biocompatible tissue of claim 34 wherein the tissue is crosslinked.

37. The biocompatible tissue of claim 36 wherein the tissue is crosslinked with a dialdehyde.

38. The biocompatible tissue of claim 36 wherein the tissue is crosslinked with a multifunctional epoxide.

39. The biocompatible tissue of claim 36 wherein the tissue is crosslinked with an epoxy amine.

40. The biocompatible tissue of claim 34 wherein the tissue comprises pericardial tissue.

41. The biocompatible tissue of claim 34 wherein the tissue comprises a multilayer composite.

42. The biocompatible tissue of claim 34 wherein the asymmetric flexibility is approximately oriented along an axis.

43. A prosthetic valve comprising the tissue of claim 34.

44. A prosthetic valve comprising a plurality of tissue leaflets wherein the tissue leaflets comprise selectively aligned tissue.

45. The prosthetic valve of claim 44 further comprising a leaflet support structure wherein the leaflets are attached to the leaflet support structure.

46. The prosthetic valve of claim 45 wherein the leaflets have less flexibility with respect to bending around axes extending from the attached edge to the free edge of the leaflet relative to bending around axes perpendicular to axes extending from the attached edge to the free edge.

47. The prosthetic valve of claim 45 wherein the leaflet support structure comprises a stent.

48. The prosthetic valve of claim 44 further comprising chordae attached to the leaflets.

49. The prosthetic valve of claim 48 wherein the leaflets have greater flexibility with respect to bending around axes extending from the attached edge of the leaflet to the chordae attachment point relative to bending around axes perpendicular to axes extending from the free edge to the chordae.

50. The prosthetic valve of claim 44 wherein the tissue comprises pericardium.

51. The prosthetic valve of claim 44 wherein the tissue is crosslinked.

52. An apparatus comprising tissue and a load applicator that applies a selected mechanical load to the tissue, wherein the load applicator comprises a gripper that grips adjacent a selected subsection of an edge of the tissue that does not approximate gripping the edge equally around the tissue.

53. The apparatus of claim 52 wherein the tissue comprises a generally flat section.

54. The apparatus of claim 52 wherein the load applicator comprises weights and connectors connecting the weights with the tissue.

55. The apparatus of claim 52 wherein the load applicator comprises a frame that grips the tissue under a load.

56. The apparatus of claim 52 wherein the load applicator comprises springs.

57. The apparatus of claim 52 wherein the load applicator comprises a motor.

58. The apparatus of claim 52 wherein the load applicator applied a load along a generally linear dimension of the tissue.

59. The apparatus of claim 52 further comprising a moisture source supplying moisture to maintain the tissue in a hydrated state.

60. The apparatus of claim 59 wherein the moisture comprises a crosslinking agent.

61. The apparatus of claim 60 wherein the crosslinking agent comprises a multifunctional composition selected from the group consisting of dialdehydes, polyepoxides and epoxyamines.
